(19) United States
(12) Patent Application Publication
(10) Pub. No.: US 2013/0030373 A1
(43) Pub. Date: Jan. 31, 2013

(54) Angiocatheter System With Anti-Leak Features

(71) Applicants: William Paul Spurbeck, Tucson, AZ (US); Mark Banister, Tucson, AZ (US); James A. Tiscione, Tucson, AZ (US)

(72) Inventors: William Paul Spurbeck, Tucson, AZ (US); Mark Banister, Tucson, AZ (US); James A. Tiscione, Tucson, AZ (US)

(73) Assignee: SFK BUSINESS VENTURES, LLC, Tucson, AZ (US)

(21) Appl. No.: 13/560,883

(22) Filed: Jul. 27, 2012

Related U.S. Application Data
(60) Provisional application No. 61/513,379, filed on Jul. 29, 2011.

(51) Int. Cl. A61M 25/16; A61B 17/34

(52) U.S. Cl. 604/167.03; 604/256

(57) ABSTRACT

Angiocatheter systems with anti-leak features for helping to prevent leaking or blood splashes that could possibly pose a health risk to the patient and/or practitioner. The systems may feature slidable anti-leak components that regulate opening and closing of the hub's hole, wherein the anti-leak components are moveable upon insertion or removal of a secondary device. The systems may feature a moveable inner wall or components on the inner wall that regulate opening of the hub's hole, wherein the wall or components of the wall are movable upon insertion or removal of a secondary device. The systems may feature compressible valves or twistable bases that regulate opening of the hub's hole, wherein the valves or bases are manipulated upon insertion or removable of a secondary device.

### CROSS REFERENCE

This application claims priority to U.S. provisional application Ser. No. 61/513,379 filed Jul. 29, 2011, the specification of which is incorporated herein by reference in its entirety.

### FIELD OF THE INVENTION

The present invention is directed to an angiocatheter system, more particularly to an angiocatheter comprising anti-leak features for helping to reduce or stop leakage or blood spill as the angiocatheter system is installed on the patient or connected to an intravenous (IV) line or other device.

### BACKGROUND OF THE INVENTION

Angiocatheters are devices that are inserted into a patient's vein for intravenous (IV) access for fluids and medications and/or for obtaining blood samples. Usually an angiocatheter is inserted by using the needle, which is positioned within the lumen of catheter. When the needle penetrates the vessel, the blood pressure in the vessel will cause blood to flow up the needle bore and into translucent tubing around the top of the angiocatheter. The practitioner verifies the penetration of the vessel by looking for blood “flash” in the chamber. The catheter of the angiocatheter is then advanced within the blood vessel to a desired position and the needle is withdrawn from the catheter.

Sometimes, for example when the needle is withdrawn and IV tubing or a blood-sampling device is introduced to the angiocatheter, blood may leak from the angiocatheter. This poses a health risk to the patient and the practitioner. The present invention features a novel angiocatheter system comprising anti-leak features that helps prevent leakage. Such a system may help provide a safer work environment for healthcare providers and technicians and a safer healthcare setting for patients.

### SUMMARY

The present invention features angiocatheter systems with anti-leak features. In some embodiments, the angiocatheter system comprises a hub (110) having a side wall (112), an inner cavity (113) accessible via an open top end (114), and a bottom portion (115), the inner cavity (113) is adapted to accept a secondary device (101), a centered first hole (119) is disposed in a bottom surface (113a) of the inner cavity (113); a chamber (120) disposed in the bottom portion (115) of the hub (110) below the bottom surface (113a) of the inner cavity (113), the chamber (120) has a side wall surface (121), a top surface (123), and a bottom surface (124), a second hole (129) is disposed in the bottom surface (124), the second hole (129) is aligned with the first hole (119) of the bottom surface (113a) of the inner cavity (113) of the hub (110); a catheter (210) fluidly connected to the second hole (129) of the chamber (120) extending through the bottom portion (115) of the hub (110) a distance past the bottom portion (115) of the hub (110); an anti-leak component (130) housed in the chamber (120), the anti-leak component (130) has a first end (131), a second end (132) opposite the first end (131), a top surface (133), and a bottom surface (134), the top surface (133) is slidably in contact with the top surface (123) of the chamber (120), the bottom surface (134) is slidably in contact with the bottom surface (124) of the chamber (120), a channel (138) is disposed in the anti-leak component (130) extending from the top surface (133) to the bottom surface (134), wherein the anti-leak component (130) is slidable between at least a closed position and an open position, in the closed position the channel (138) is un-aligned with the first hole (119) in the bottom surface (113a) of the inner cavity (113) of the hub (110) and the second hole (129) in the chamber (120), in the open position the channel (138) is aligned with the first hole (119) in the bottom surface (113a) of the inner cavity (113) of the hub (110) and the second hole (129) in the chamber (120), when the anti-leak component (130) is in the open position fluid can flow between the catheter (210) and the inner cavity (113) of the hub (110), the anti-leak component (130) is biased in the closed position via a biasing mechanism; and a resetting mechanism for moving the anti-leak component (130) to the open position.

In some embodiments, the biasing mechanism functions to push the anti-leak component (130) to un-align the channel (138) with the first hole (119) and second hole (129). In some embodiments, the biasing mechanism comprises a spring (140) that functions to push the anti-leak component (130) away from the side wall surface (121) of the chamber (120) thereby un-aligning the channel (138) with the first hole (119) and the second hole (129). In some embodiments, the resetting mechanism comprises a pivot lever (150) disposed in a pivot lever cavity (155) disposed in the side wall (112) of the hub (110), the pivot lever (150) has a first end (151) and a second end (152), the first end (151) is adapted to engage the inner cavity (113) of the hub (110), the second end (152) is adapted to engage the second end (132) of the anti-leak component (130), the pivot lever (150) can pivot between a first position and a second position, in the first position the first end (151) is at least partially pressed into the inner cavity (113) of the hub (110) via an entrance (158) disposed in a inner surface (113b) of the inner cavity (113), the entrance (158) being either a membrane or a hole, in the second position the second end (152) of the pivot lever (150) presses against the second end (132) of the anti-leak component (130) thereby moving the anti-leak component (130) to the open position, the pivot lever (150) is biased in the first position caused by the spring (140) biasing the anti-leak component (130) to the closed position.

In some embodiments, the resetting mechanism is activated upon insertion of a secondary device (101) into the inner cavity (113) of the hub (110). In some embodiments, insertion of a secondary device (101) into the inner cavity (113) of the hub (110) moves the pivot lever (150) to the second position, which moves the anti-leak component to the open position.

In some embodiments, the resetting mechanism comprises a button (160) disposed on the outer surface of the hub (110). In some embodiments, the button (160) engages the second end (132) of the anti-leak component (130), when the button (160) is pressed the anti-leak component (130) is moved to the first position. In some embodiments, the button (160) can be locked in the pressed position via a locking system (250).

In some embodiments, the resetting mechanism comprises a wedge (170) having a pointed end, a flat end, and a hypotenuse end, the pointed end is disposed in the chamber (120) and the hypotenuse end contacts the second end (132) of the anti-leak component (130), the flat end can extend upwardly through the bottom surface (113a) of the inner cavity (113) of the hub (110), the wedge (170) can move between a first position and a second position, in the first position the wedge (170) is pushed upwardly into the inner cavity (113) of the hub (110) via the pressure of the spring (140) and the anti-leak component (130) such that the anti-leak component (130) occupies the closed position, in the second position the wedge (170) is pushed downwardly into the chamber (120) and the hypotenuse end of the wedge (170) presses against the second end (132) of the anti-leak component (130) thereby moving the anti-leak component (130) to the open position, the wedge (170) is biased in the first position caused by the spring (140) biasing the anti-leak component (130) in the closed position. In some embodiments, the wedge (170) is compressible. In some embodiments, insertion of a secondary device (101) into the inner cavity (113) of the hub (110) pushes the wedge (170) to the second position, which moves the anti-leak component to the open position.

In some embodiments, the system further comprises a needle (103) removably housed in the catheter (210), the needle (103) can be withdrawn from the catheter (210). In some embodiments, the system further comprises a needle (103) removably housed in the catheter (210), the needle can be withdrawn from the catheter (210), wherein removal of the needle (103) results in the anti-leak valve moving to the closed position to prevent flow from the catheter (210) to the inner cavity (113) of the hub (110). In some embodiments, the system further comprises a spacer component (240) disposed on at least a portion of the bottom surface (113a) of the inner cavity (113) of the hub (110).

In some embodiments, the angiocatheter system comprises a hub (110) having an inner cavity (113) accessible via an open top end (114), and a bottom portion (115), the inner cavity (113) is adapted to accept a secondary device (101), a centered first hole (119) is disposed in a bottom surface (113a) of the inner cavity (113); a catheter (210) extending from a second hole (129) in the bottom portion (115) of the hub (110) a distance past the bottom portion (115) of the hub (110), the first hole (119) and the second hole (129) are aligned; an anti-leak component (130) housed in the bottom portion (115) of the hub (110), the an anti-leak component (130) can move between at least a closed position and an open position, in the closed position fluid is prevented from flowing between the catheter (210) and the first hole (119) in the hub (110), in the open position fluid can flow between the catheter (210) and the first hole (119) in the hub (110), the anti-leak component (130) is biased in the closed position via a biasing mechanism; and a resetting mechanism for moving the anti-leak component (130) to the open position.

In some embodiments, the system further comprises a chamber (120) disposed in the bottom portion (115) of the hub (110) below the bottom surface (113a) of the inner cavity (113), the chamber (120) has a side wall surface (121), a top surface (123), and a bottom surface (124), the second hole (129) is disposed in the bottom surface (124), wherein the anti-leak component (130) is housed in the chamber (120), the anti-leak component (130) comprises a channel (138) extending from a top surface (133) to a bottom surface (134), the top surface (133) of the anti-leak component (130) is slidably in contact with the top surface (123) of the chamber (120), the bottom surface (134) of the anti-leak component (130) is slidably in contact with the bottom surface (124) of the chamber (120), wherein in the closed position the channel (138) is un-aligned with the first hole (119) in the bottom surface (113a) of the inner cavity (113) of the hub (110) and the second hole (129) in the chamber (120), in the open position the channel (138) is aligned with the first hole (119) in the bottom surface (113a) of the inner cavity (113) of the hub (110) and the second hole (129) in the chamber (120).

In some embodiments, the biasing mechanism comprises a spring (140) that engages a first end (131) of the anti-leak component and a side wall surface (121) of the chamber (120) and pushes the anti-leak component away from the side wall surface (121) of the chamber (120) thereby un-aligning the channel (138) with the first hole (119) and the second hole (129).

In some embodiments, the resetting mechanism comprises a pivot lever (150) disposed in the side wall (111) of the hub (110), the pivot lever (150) has a first end (151) and a second end (152), the first end (151) is adapted to engage the inner cavity (113) of the hub (110), the second end (152) is adapted to engage the second end (132) of the anti-leak component (130), the pivot lever (150) can pivot between a first position and a second position, in the first position the first end (151) is at least partially pressed into the inner cavity (113) of the hub (110) either via a membrane disposed in a inner surface (113b) of the inner cavity (113) or via a side hole disposed in the inner surface (113b) of the inner cavity (113), in the second position the second end (152) of the pivot lever (150) presses against the second end (132) of the anti-leak component (130) thereby moving the anti-leak component (130) to the open position, the pivot lever (150) is biased in the first position caused by the biasing of the anti-leak component (130) to the closed position.

In some embodiments, the resetting mechanism is activated upon insertion of a secondary device (101) into the inner cavity (113) of the hub (110). In some embodiments, insertion of a secondary device (101) into the inner cavity (113) of the hub (110) moves the pivot lever (150) to the second position, which moves the anti-leak component to the open position.

In some embodiments, the resetting mechanism comprises a button disposed on the outer surface of the hub (110). In some embodiments, the button engages the second end (132) of the anti-leak component (130), when the button is pressed the anti-leak component (130) is moved to the first position.

In some embodiments, the resetting mechanism comprises a wedge having a pointed end, a flat end, and a hypotenuse end, the pointed end is disposed in the chamber (120) and the hypotenuse end contacts the second end (132) of the anti-leak component (130), the flat end can extend upwardly through the bottom surface (113a) of the inner cavity (113) of the hub (110), the wedge can move between a first position and a second position, in the first position the wedge is pushed upwardly into the inner cavity (113) of the hub (110) via the pressure of the spring (140) and the anti-leak component (130) such that the anti-leak component (130) occupies the closed position, in the second position the wedge is pushed downwardly into the chamber (120) and the hypotenuse end of the wedge presses against the second end (132) of the anti-leak component (130) thereby moving the anti-leak component (130) to the open position, the wedge is biased in the first position caused by the spring (140) biasing the anti-leak component (130) in the closed position. In some embodiments, insertion of a secondary device (101) into the inner cavity (113) of the hub (110) pushes the wedge to the second position, which moves the anti-leak component to the open position.

In some embodiments, the system further comprises a needle removably housed in the catheter (210), the needle can be withdrawn from the catheter (210). In some embodiments, the system further comprises a needle removably housed in the catheter (210), the needle can be withdrawn from the catheter (210), wherein removal of the needle results in the anti-leak valve moving to the closed position to prevent flow from the catheter (210) to the inner cavity (113) of the hub (110). In some embodiments, the system further comprises a compressible material disposed on at least a portion of the bottom surface (113a) of the inner cavity (113) of the hub (110).

In some embodiments, the angiocatheter system comprises a hub (110) having a side wall (112), an inner cavity (113) accessible via an open top end (114), and a bottom portion (115), the inner cavity (113) has a bottom surface (113a) and a wall (113b), the inner cavity (113) is adapted to engage a secondary device (101), a centered first hole (119) is disposed in the bottom surface (113a) of the inner cavity (113); and a catheter (210) fluidly connected to the first hole (119) of the hub (110) extending through the bottom portion (115) of the hub (110) a distance past the bottom portion (115) of the hub (110); wherein the wall (113b) of the inner cavity (113) can expand to a first position when a secondary device (101) engages the inner cavity (113) and contract to a second position when a secondary device (101) is removed from the inner cavity (113), when the wall (113b) of the inner cavity (113) is in the first position the first hole (119) is open allowing flow between the catheter (210) and the Secondary device (101), when the wall (113b) of the inner cavity (113) is in the second position the first hole (119) is closed preventing flow from the catheter (210) and the Secondary device (101), the wall (113b) of the inner cavity (113) is biased in the second position.

In some embodiments, the bottom surface (113a) of the inner cavity (113) is stretched when the wall (113b) of the inner cavity (113) is expanded to the first position. In some embodiments, the bottom surface (113) is constructed from a material comprising rubber, elastic, latex, or a combination thereof. In some embodiments, the bottom surface (113a) of the inner cavity (113) biases the wall (113b) of the inner cavity (113) in the second position. In some embodiments, the wall (113b) of the inner cavity (113) is constructed from a plurality of concave blades (113c), the concave blades (113c) have a narrow end (116c) and a wide end (116d), the narrow ends (116c) together form the first hole (119) and the bottom surface (113a) of the inner cavity (113). In some embodiments, the concave blades are concave trapezoidal blades. In some embodiments, the wall (113b) of the inner cavity (113) comprises a first angled flange (310a) and a second angled flange (310b) opposite the first angled flange, the angled flanges extend into the inner cavity (113) of the hub (110), each angled flange has a narrow end and a wide end and can slide inwardly to a first position and outwardly to a second position with respect to the inner cavity (113), when in the first position the wide end of the first angled flange overlaps the wide end of the second angled flange thereby blocking the hole (119), when in the second position the wide ends of the angled flanges are slid away from the first hole (119) to allow access to the first hole (119). In some embodiments, only a portion of the wall of the inner cavity (113) is expandable. In some embodiments, a bottom portion of the wall of the inner cavity (113) is expandable.

In some embodiments, the angiocatheter system comprises a hub (110) having a side wall (112), an inner cavity (113) accessible via an open top end (114), and a bottom portion (115), the inner cavity (113) has a bottom surface (113a) and a wall (113b), the inner cavity (113) is adapted to engage a secondary device (101), a centered first hole (119) is disposed in the bottom surface (113a) of the inner cavity (113); a catheter (210) fluidly connected to the first hole (119) of the hub (110) extending through the bottom portion (115) of the hub (110) a distance past the bottom portion (115) of the hub (110); a base (180) disposed in the inner cavity (113) at its bottom surface (113a), the base (180) is cylindrical having a center slit (182) disposed therein and a plurality of ribs (190) radially extending from the center slit (182), the base (180) occupies a closed position wherein the slit (182) is un-aligned with the first hole (119) in the bottom surface (113a) of the inner cavity (113) preventing flow between the inner cavity (113) and the catheter (210), wherein upon insertion of a secondary device (101) into the inner cavity (113) of the hub (110) the base (180) is compressible and the base (180) rotates in a first direction to an open position such that the slit (182) is aligned with the first hole (119) in the bottom surface (113a) of the inner cavity (113) allowing flow between the catheter (210) and the inner cavity (113).

In some embodiments, the ribs are constructed from a material comprising rubber, elastic, plastic, latex, or a combination thereof.

In some embodiments, the angiocatheter system comprises a catheter 1910 having a distal end and a proximal end; and an anti-leak component 1950 permanently disposed on the proximal end of the catheter 1910, the anti-leak component has a receiving end adapted to engage a secondary device, wherein an aperture 1950 is disposed in the receiving end of the anti-leak component 1950, wherein the anti-leak component 1950 can move between at least an open position and a closed position respectively allowing and preventing flow through the catheter 1910, the anti-leak component 1950 is biased in the closed position and can be moved to the open position upon engagement of the secondary device with the receiving end of the anti-leak component 1950.

In some embodiments, the system further comprises a needle 1918 removably housed in the catheter 1910 and in the aperture 1956 of the anti-leak component 1910, the needle 1918 can be withdrawn from the catheter 1910 through the anti-leak component 1910, wherein removal of the needle 1918 causes the anti-leak valve 1950 to occupy the closed position to prevent flow through the catheter 1910. In some embodiments, the receiving end of the anti-leak component 950 is a female-shaped adaptor. In some embodiments, the secondary device is a syringe or IV line. In some embodiments, the anti-leak component is a valve. In some embodiments, the valve is compressible.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

FIG. 1A is a front cross sectional view of the system of the present invention. The needle is housed in the catheter (prior to removal of the needle). The anti-leak component is in the open position. The resetting mechanism is not shown.

FIG. 1B is a front cross sectional view of the system of the present invention. The needle is housed in the catheter (prior to removal of the needle). The anti-leak component is in the open position. The resetting mechanism is not shown.

FIG. 1C is a front cross sectional view of the system of the present invention. The anti-leak component is in the open position. The resetting mechanism is not shown.

FIG. 1D is a front cross sectional view of the system of the present invention. The anti-leak component is in the closed position. The resetting mechanism is not shown.

FIG. 1E is a front cross sectional view of an embodiment of the hub of the system of the present invention. The anti-leak component, chamber, and resetting mechanism are not shown. The side wall of the hub is shown as a cavity. The wall of the hub may be a cavity (e.g. hollow), may be a solid wall, or may be partially hollow. The present invention is not limited to these embodiments.

FIG. 1F is a front cross sectional view of an embodiment of the hub of the system of the present invention. The side wall of the hub is shown as a solid wall. The wall of the hub may be a cavity (e.g. hollow), may be a solid wall, or may be partially hollow. The present invention is not limited to these embodiments.

FIG. 2 is a detailed front cross sectional view of FIG. 1C.

FIG. 3A is a detailed front cross sectional view of the system of the present invention. The resetting mechanism shown is the pivot lever. The anti-leak component is in the closed position.

FIG. 3B is a detailed front cross sectional view of the system of the present invention. The resetting mechanism shown is the pivot lever. The anti-leak component is in the open position.

FIG. 3C is a detailed front cross sectional view of FIG. 3A. The resetting mechanism shown is the pivot lever. The Secondary device is about to be inserted. The anti-leak component is still in the closed position.

FIG. 3D is a detailed front cross sectional view of FIG. 3B. The resetting mechanism shown is the pivot lever. The Secondary device is inserted, and the anti-leak component is in the open position.

FIG. 4A is a detailed front cross sectional view of the system of the present invention. The resetting mechanism shown is the button. The anti-leak component is in the closed position.

FIG. 4B is a detailed front cross sectional view of the system of the present invention. The resetting mechanism shown is the button. The anti-leak component is in the open position.

FIG. 4C is a detailed front cross sectional view of FIG. 4A. The resetting mechanism shown is the button. The Secondary device is about to be inserted. The anti-leak component is still in the closed position.

FIG. 4D is a detailed front cross sectional view of FIG. 4B. The resetting mechanism shown is the button. The Secondary device is inserted, and the anti-leak component is in the open position.

FIG. 5A is a detailed front cross sectional view of the system of the present invention. The resetting mechanism shown is the wedge. The anti-leak component is in the closed position.

FIG. 5B is a detailed front cross sectional view of the system of the present invention. The resetting mechanism shown is the wedge. The anti-leak component is in the open position.

FIG. 5C is a detailed front cross sectional view of FIG. 5A. The resetting mechanism shown is the wedge. The Secondary device is about to be inserted. The anti-leak component is still in the closed position.

FIG. 5D is a detailed front cross sectional view of FIG. 5B. The resetting mechanism shown is the wedge. The Secondary device is inserted, and the anti-leak component is in the open position.

FIG. 6A is a top view of the inner cavity of the system of the present invention.

FIG. 6B is a top view of the chamber of the system of the present invention. The anti-leak component is in the closed position (covering the second hole). The resetting mechanism is not shown.

FIG. 7A is a front cross sectional view of the system of the present invention. The wall of the inner cavity is compressed, which compresses the bottom surface of the inner cavity thereby closing the hole. The resetting mechanism is not shown.

FIG. 7B is a front cross sectional view of the system of the present invention. The wall of the inner cavity is expanded, which expands the bottom surface of the inner cavity thereby opening the hole. The resetting mechanism is not shown.

FIG. 7C is a front cross sectional view of FIG. 7A. The Secondary device is about to be inserted. The hole is still closed.

FIG. 7D is a front cross sectional view of FIG. 7B. The IV tubing is inserted, and the hole is open.

FIG. 7E is an alternative embodiment of the system shown in FIG. 7A. The bottom portion of the wall of the inner cavity is expandable and regulates expansion of the bottom surface of the inner cavity (and opening of the hole). The IV tubing is about to be inserted. The hole is still closed.

FIG. 8A is a top view of the expandable bottom surface of the inner cavity. The hole is closed.

FIG. 8B is a top view of the expandable bottom surface of the inner cavity. The hole is open.

FIG. 8C is a top view of the expandable bottom surface of the inner cavity. Ribs are present in the bottom surface for structural support. The hole is open.

FIG. 9A is an alternative embodiment of the system of the present invention. Two opposing flanges can be opened and closed to regulate opening and closing of the hole. The hole is open.

FIG. 9B is an alternative embodiment of the system of the present invention. Two opposing flanges can be opened and closed to regulate opening and closing of the hole. The flanges overlap to close the hole.

FIG. 10A is a front cross sectional view of the system of FIG. 9A. The flanges are separated to open the hole.

FIG. 10B is a front cross sectional view of the system of FIG. 9B. The flanges overlap to close the hole.

FIG. 11A is a front cross sectional view of the system of the present invention. The base with ribs and slit is disposed in the inner cavity. The hole is effectively closed (the base is positioned above the cone).

FIG. 11B is a front cross sectional view of the system of the present invention. The base with ribs and slit is disposed in the inner cavity. The hole is effectively opened (the base is pushed downwardly over the cone, opening the hole).

FIG. 12 is a top view of the system of FIG. 11.

FIG. 13A is a side cross sectional view of an angiocatheter system of the present invention.

FIG. 13B is an exploded view of the system of FIG. 13A.

FIG. 13C is a side cross sectional view of an embodiment of the angiocatheter system of the present invention.

FIG. 13D is an exploded view of the system of FIG. 13C.

FIG. 14 is a side cross sectional view of an embodiment of the angiocatheter system of the present invention. The compressible valve is in the extended position.

FIG. 15 is a side cross sectional view of an embodiment of the angiocatheter system of the present invention, wherein a syringe is partially inserted into the hub and the compressible valve is partially compressed.

FIG. 16 is a side cross sectional view of an embodiment of the angiocatheter system of the present invention, wherein a syringe is partially inserted into the hub and the compressible valve is partially compressed.

FIG. 17 is a side cross sectional view of an embodiment of the angiocatheter system of the present invention, wherein a syringe is inserted into the hub and the compressible valve is in the compressed position.

FIG. 18 is a side view of an embodiment of the angiocatheter system of the present invention.

FIG. 19 is a side view of an embodiment of the angiocatheter system of the present invention comprising a reed-type valve.

FIG. 20A is a side view of an embodiment of the angiocatheter system of the present invention comprising a tines valve.

FIG. 20B is an end view of the tines valve of FIG. 20A in the closed position.

FIG. 21A is a side view of an embodiment of the angiocatheter system of the present invention comprising a clapper-type valve.

FIG. 21B is a side view of the system of FIG. 21A before the needle and needle stick prevention device are removed.

FIG. 22A is a side view of an embodiment of the angiocatheter system of the present invention, wherein an end cap is attached.

FIG. 22B is a side view of the system of FIG. 22A wherein a saline lock is inserted into the connection hub.

FIG. 23A is a side view of an embodiment of the angiocatheter system of the present invention comprising a septum-type valve that closes when the needle is pulled out and can re-open when a standard male Luer lock is attached by pushing the septum valve over the stationary anchor.

FIG. 23B is a side and end views of the septum valve of the system of FIG. 23A.

### DESCRIPTION OF PREFERRED EMBODIMENTS

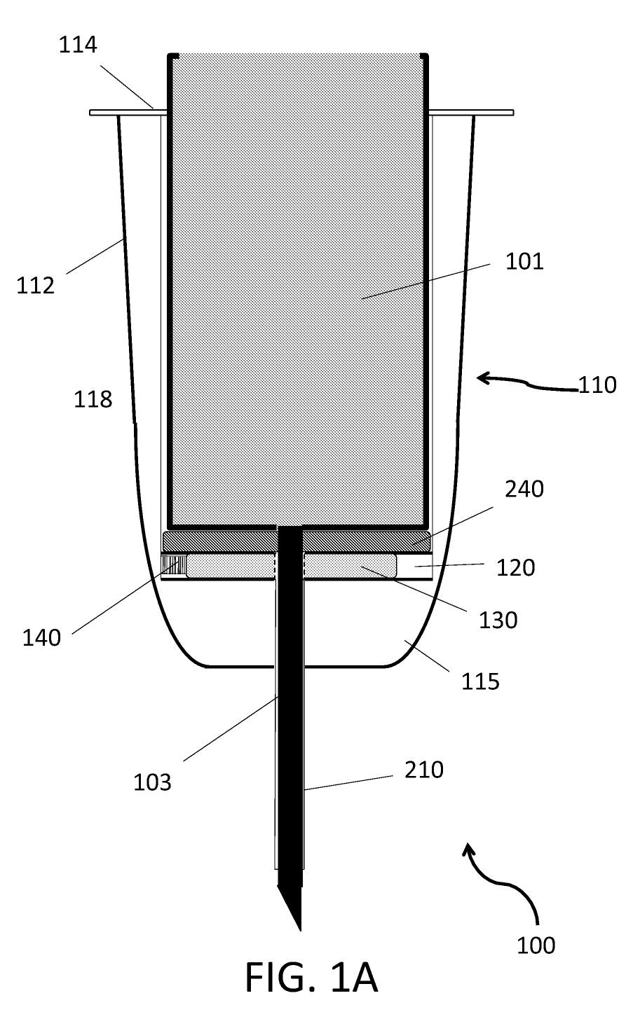

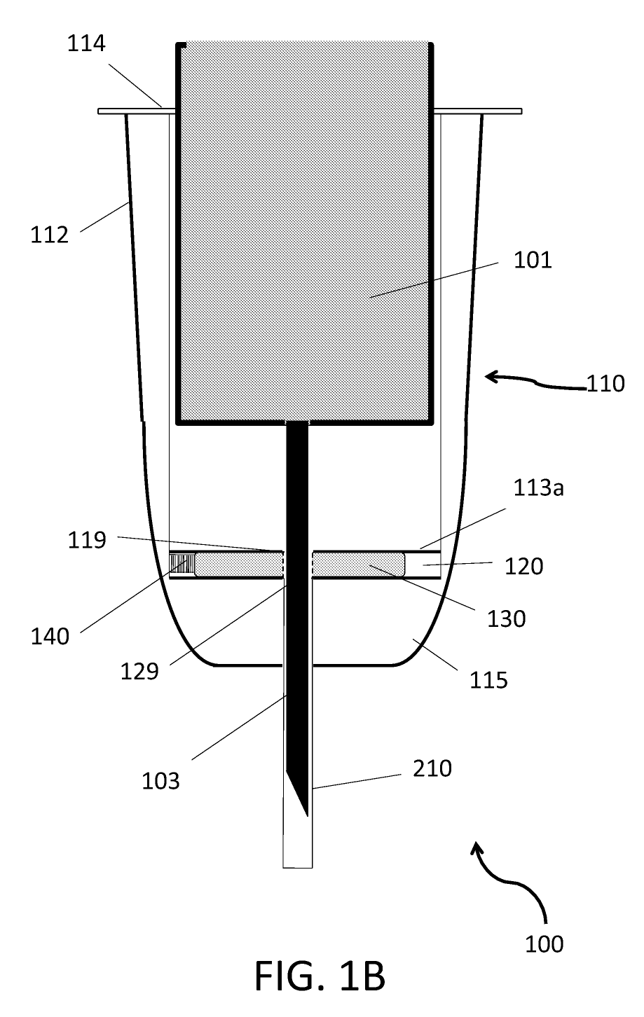

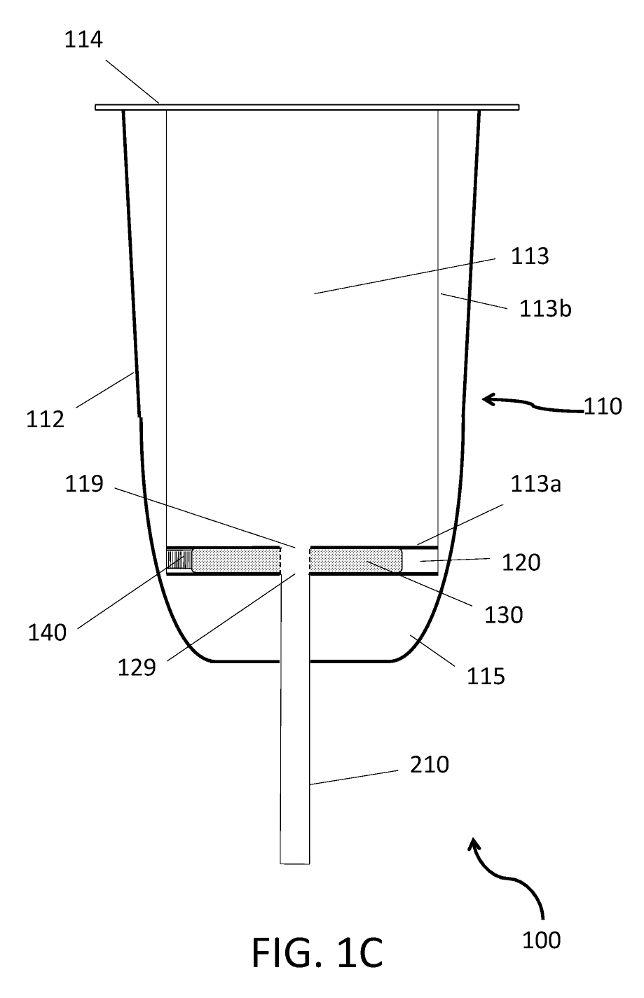

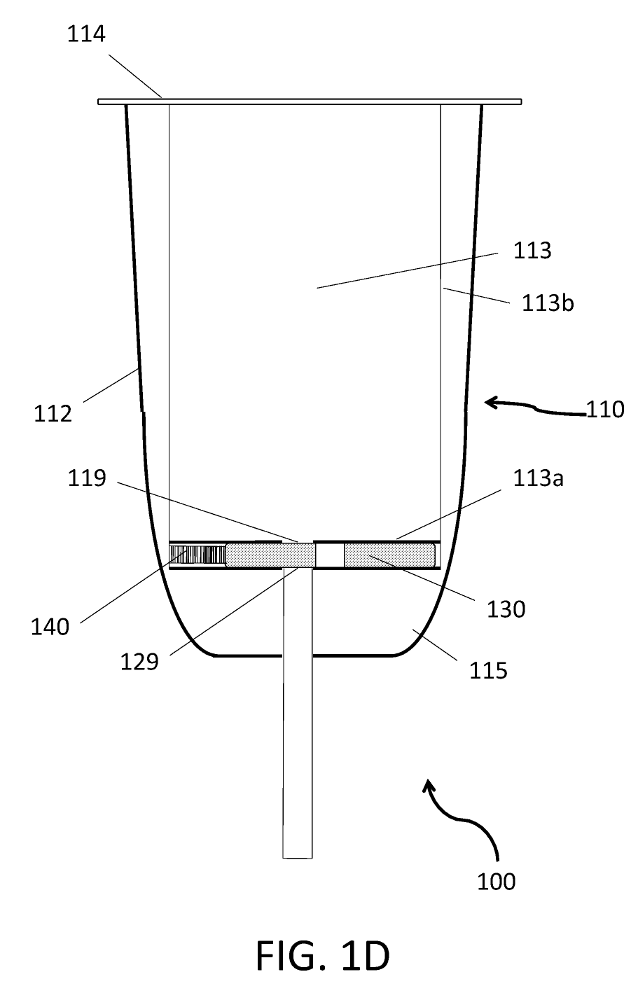

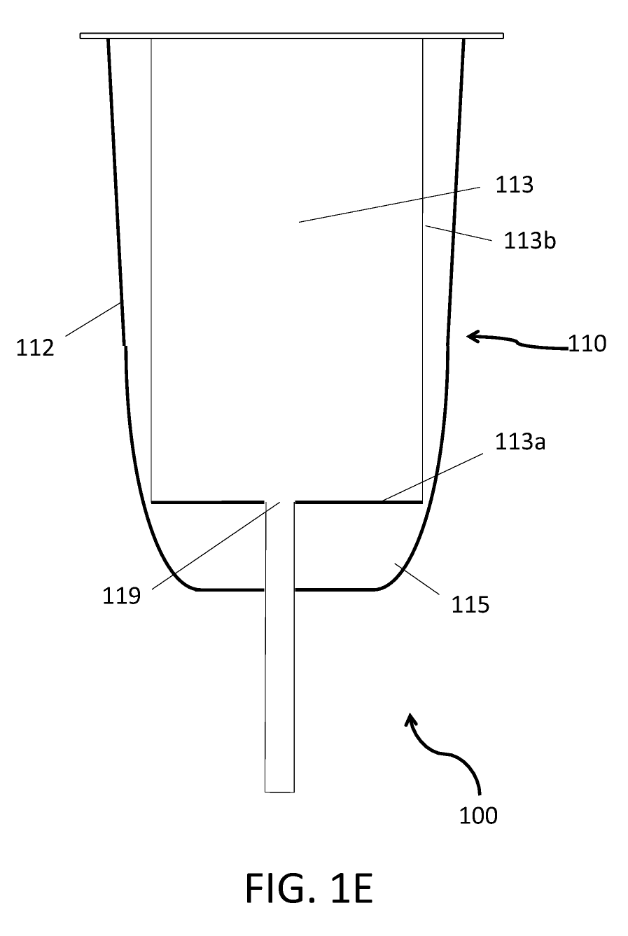

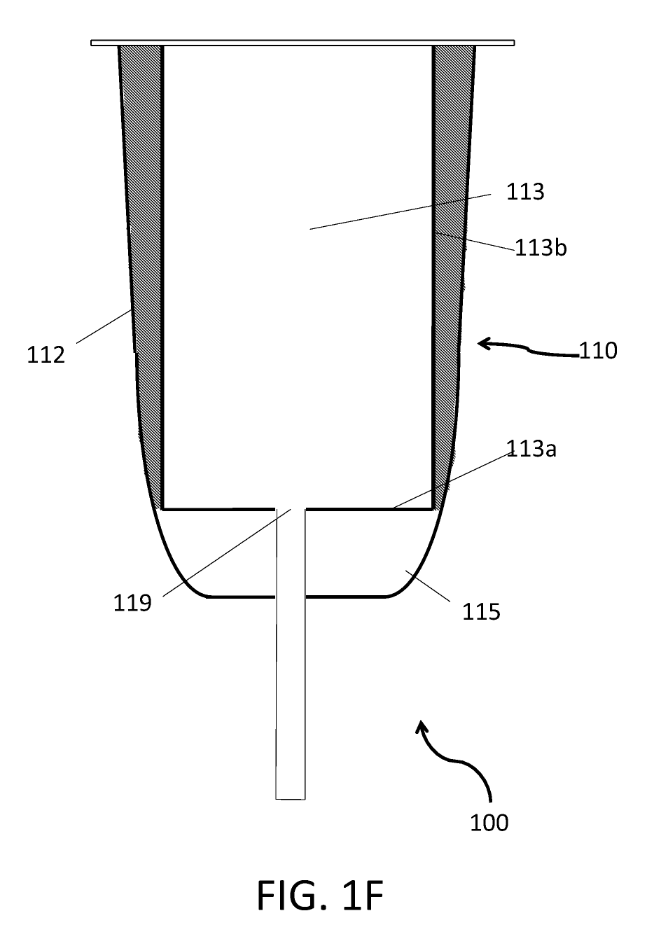

Referring now to FIG. 1-23, the present invention features an angiocatheter system (100) comprising anti-leak features. The anti-leak features may help prevent leaking or blood splashes that could possibly pose a health risk to the patient and/or practitioner.

As used herein, the term “catheter” (e.g., catheter 210) refers to the sheath or tubing portion of the system (100) that covers the needle (103) and is left in the vein once the needle (103) is removed. The catheter (210) may also be referred to as a “cannula.” Catheters (cannulas) are standard features of standard angiocatheters and are well known to one or ordinary skill in the art.

As used herein, the terms “secondary device” and “IV tubing component” refer to any device appropriate for insertion into the hub, for example a Luer lock device, a syringe, etc.

System with Movable Anti-Leak Component

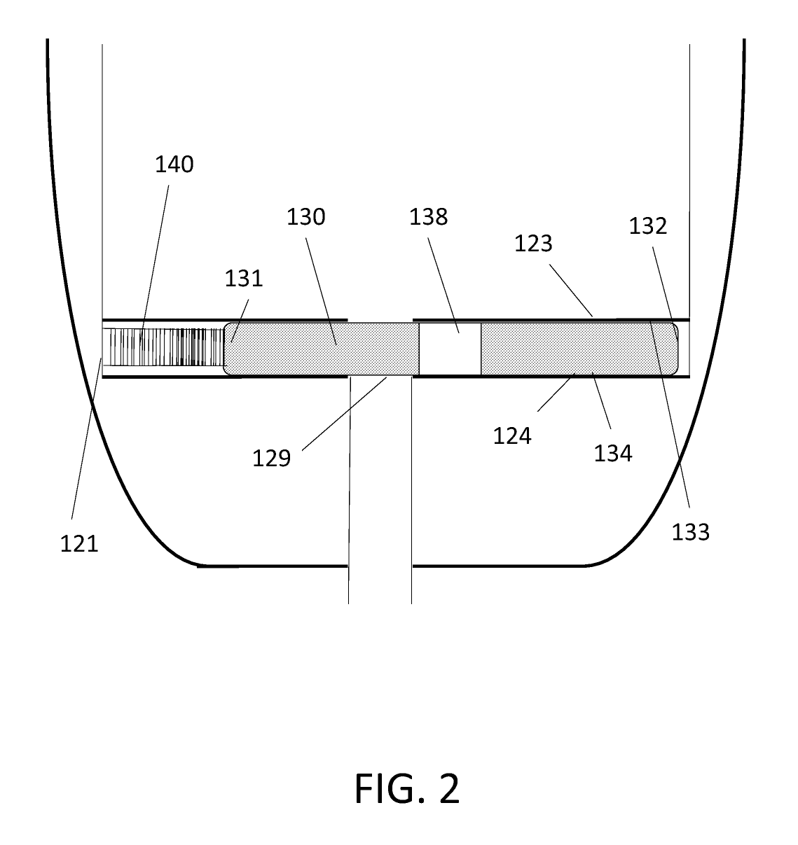

Referring to FIG. 1A-1F and FIG. 2, the system (100) of the present invention comprises a hub (110) and a catheter (210) that extends from the hub (110). The hub (110) is a receiving end (a female receptacle) for a secondary device (101), e.g., an intravenous tubing component such as an IV line, a syringe, etc. The hub (110) has a side wall (112), an inner cavity (113), an open top end (114), and a bottom portion (115). The inner cavity (113) is accessible vie the open top end (114), e.g., the secondary device (101) (IV tubing component) can be inserted into the inner cavity (113) via the open top end (114).

The wall (112) of the hub (110) may comprise cavities, be hollow, be partially hollow, or be solid (e.g., see FIG. 1E-F). The present invention is not limited to these configurations. For example, while the wall (112) in FIG. 1F is shown as a solid wall, any of the other embodiments shown may also comprise a wall (112) that is solid. As such, any of the embodiments may comprise a wall (112) that comprises a cavity or is partially hollow, or is wholly hollow.

The inner cavity (113) is adapted to accept a secondary device (101), e.g., IV tubing component. The secondary device (101) may feature a Luer system such as a Luer lock or a Luer Slip system. Luer systems are well known to one of ordinary skill in the art and are commonly found on secondary devices. In some embodiments, the top end (114) of the hub (110) engages the Luer system of the secondary device.

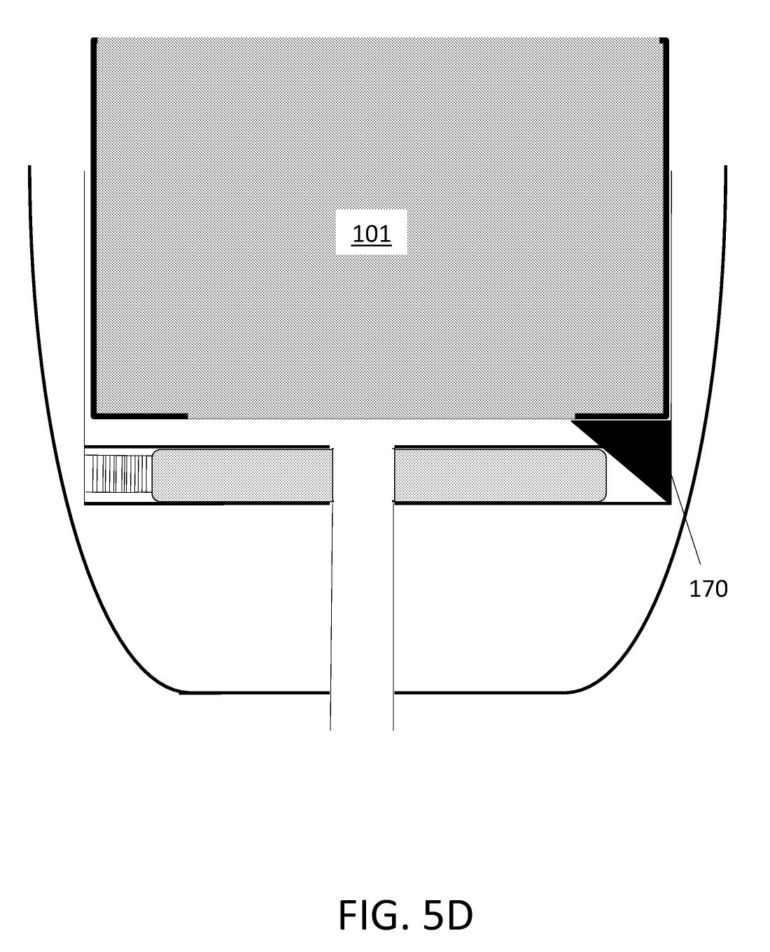

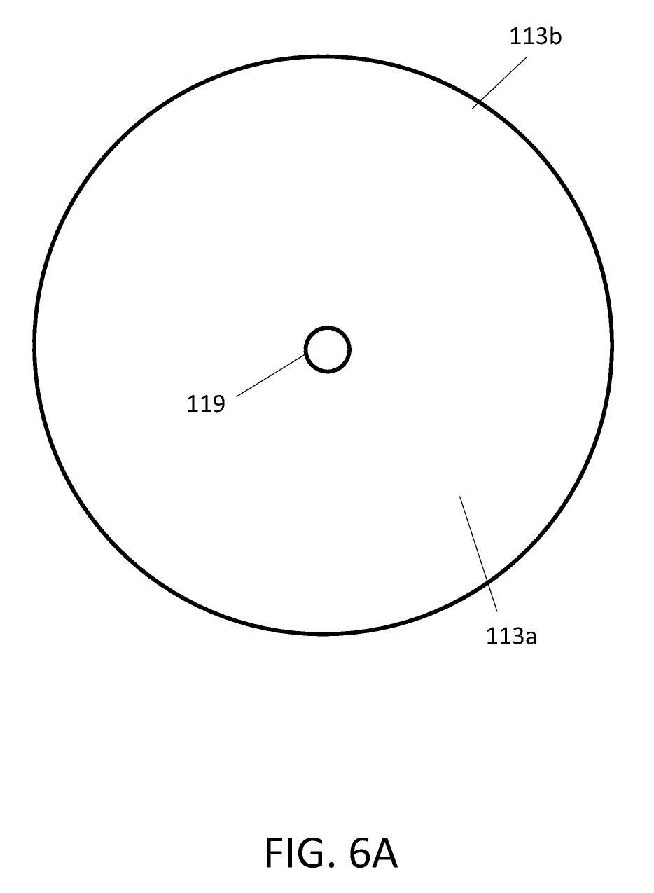

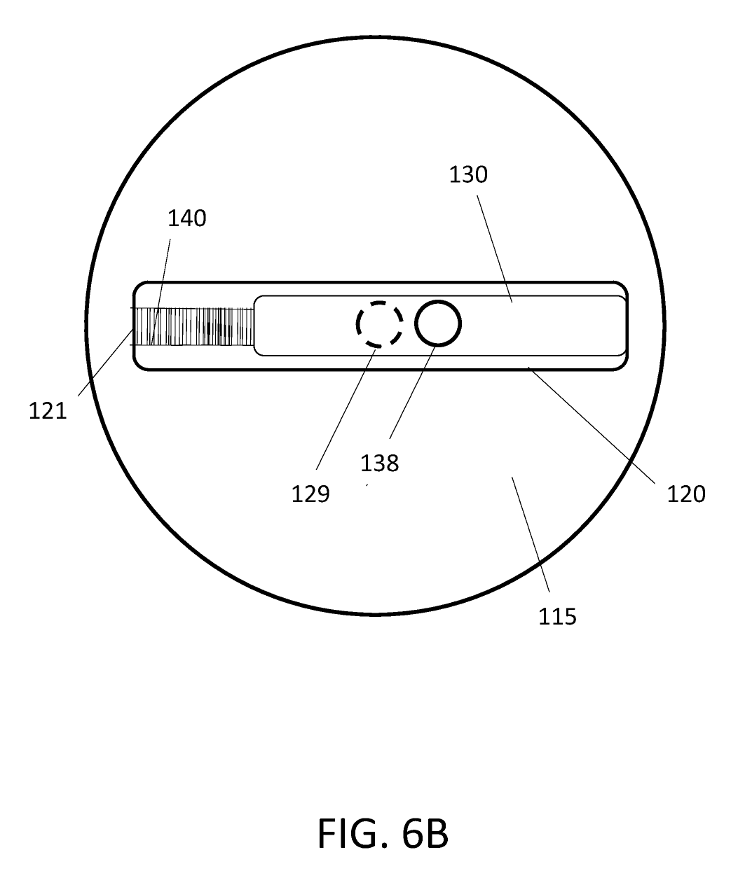

A first hole (119) is disposed in a bottom surface (113a) of the inner cavity (113) (see also FIG. 6A). The first hole (119) may be centered in the bottom surface (113a) of the inner cavity (113). The hole (119) is adapted to accept a needle (103), e.g., a needle (103) that temporarily resides in the catheter (210) that extends from the hub (110).

In some embodiments, an anti-leak component (130) is disposed in the bottom portion (115) of the hub (110), for example below the bottom surface (113a) of the inner cavity (113). The anti-leak component (130) may be housed in a chamber (120). For example, referring to FIG. 1A-1F, FIG. 2, and FIG. 6B, in some embodiments, a chamber (120) is disposed in the bottom portion (115) of the hub (110) below the bottom surface (113a) of the inner cavity (113). An anti-leak component (130) is housed in the chamber (120).

The chamber (120) has a side wall surface (121), a top surface (123), and a bottom surface (124). A second hole (129) is disposed in the bottom surface (124) of the chamber (120). The second hole (129) may optionally be centered with respect to the bottom surface (124) of the chamber (120), depending on the design of the chamber (120). The second hole (129) is aligned with the first hole (119) in the bottom surface 9113a) of the inner cavity (113) of the hub (110). The second hole (129) is adapted to accept a needle (103), e.g., a needle (103) that temporarily resides in the catheter (210) that extends from the hub (110).

In some embodiments, the catheter (210) is fluidly connected to the second hole (129) of the chamber (120). The catheter (210) extends from the second hole (129) through the bottom portion (115) of the hub (110) a distance past the bottom portion (115) of the hub (110). In some embodiments, the system (100) comprises a needle (103) temporarily sheathed by the catheter (210).

In some embodiments, an anti-leak component (130) is housed in the chamber (120). As shown in FIG. 1A-1F, FIG. 2, and FIG. 6B, the anti-leak component (130) has a first end (131), a second end (132) opposite the first end (131), a top surface (133), and a bottom surface (134). A channel (138) is disposed in the anti-leak component (130) extending from the top surface (133) to the bottom surface (134). The channel (138), like the first hole (119) and second hole (129), is adapted to accept a needle (103), e.g., a needle (103) that temporarily resides in the catheter (210) that extends from the hub (110).

The top surface (133) is slidably in contact with the top surface (123) of the chamber (120), and the bottom surface (134) is slidably in contact with the bottom surface (124) of the chamber (120). The contact between the top surface (133) of the anti-leak component (130) and the top surface (123) of the chamber (120) and the contact between the bottom surface (134) of the anti-leak component (130) and the bottom surface (124) of the chamber (120) can help create a temporary seal and help prevent fluid flow from the holes (119, 129) and/or channel (138) into the chamber (120).

The anti-leak component (130) can slide within the chamber (120) between at least a closed position and an open position. In the closed position, as shown in FIG. 1C and FIG. 2, the channel (138) of the anti-leak component (130) is un-aligned with the first hole (119) in the bottom surface (113a) of the inner cavity (113) of the hub (110) and the second hole (129) in the chamber (120). This prevents flow between the catheter (210) and the inner cavity (113) of the hub (110) and/or the IV tubing component (101).

In the open position, as shown in FIG. 1A-C, the channel (138) of the anti-leak component (130) is aligned with the first hole (119) in the bottom surface (113a) of the inner cavity (113) of the hub (110) and the second hole (129) in the chamber (120). When the anti-leak component (130) is in the open position, fluid can flow between the catheter (210) and the inner cavity (113) of the hub (110) and/or secondary device (101).

The anti-leak component (130) is biased in the closed position via a biasing mechanism. For example, in some embodiments the biasing mechanism comprises a spring (140) disposed on the side wall surface (121) of the chamber (120). The spring (140) engages the first end (131) of the anti-leak component and pushes the anti-leak component away from the side wall surface (121) of the chamber (120) (e.g., in the opposite direction), thereby un-aligning the channel (138) with the first hole (119) and the second hole (129). The present invention is not limited to a spring for a biasing mechanism.

In some embodiments, the system (100) comprises a needle (103) temporarily housed in the catheter (210) (see FIG. 1A-B). In some embodiments, the needle (103) can be withdrawn from the catheter (210). In some embodiments, removal of the needle (103) results in the anti-leak component (130) moving to the closed position to prevent flow between the inner cavity (113) of the hub (110) and the catheter (210).

The anti-leak component (130) may be any appropriate component that allows for temporary stoppage of flow between the catheter (210) and inner cavity (113) of the hub (110) and/or the secondary device (101). For example, in some embodiments, the anti-leak component (130) is a valve, e.g., a compressible valve. Likewise, the anti-leak component (130) may be constructed in a variety of shapes, e.g., rectangular as shown in FIG. 6B. However, the anti-leak component (130) is not limited to a rectangular shape and may be constructed in an appropriate shape, e.g., oval, circular, etc.

In some embodiments, a spacer component (240), e.g., a foam, sponge, a compressible rubber, etc., (e.g., a compressible material), may be disposed on the bottom surface (113a) of the inner cavity (113). In some embodiments, the spacer component (240) helps close off any space in between the secondary device (101), e.g., IV tubing component, and the bottom surface (113a) of the inner cavity (113) when the secondary device (101) is inserted (and/or properly placed). The spacer component (240) may be disposed on a portion of the bottom surface (113a) of the inner cavity (113). The spacer component (240) may comprise a channel through which fluid can flow between the catheter (210) and the secondary device (101).

The system (100) of the present invention further comprises a resetting mechanism for moving the anti-leak component (130) to the open position. In some embodiments, the resetting mechanism is activated upon insertion (and optionally proper placement) of a secondary device (101), e.g., an IV tubing component, into the inner cavity (113) of the hub (110).

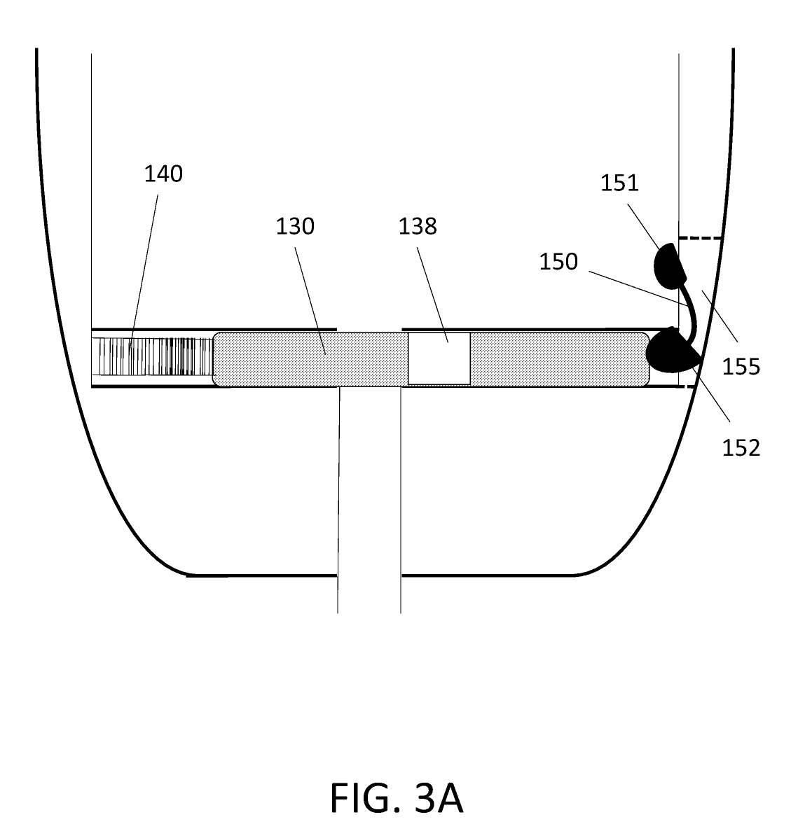

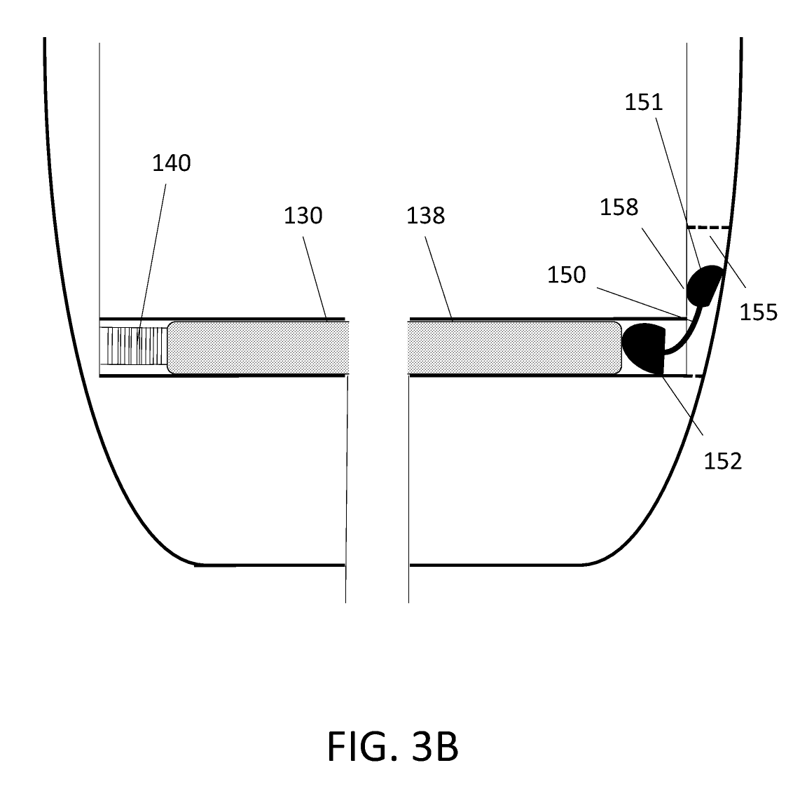

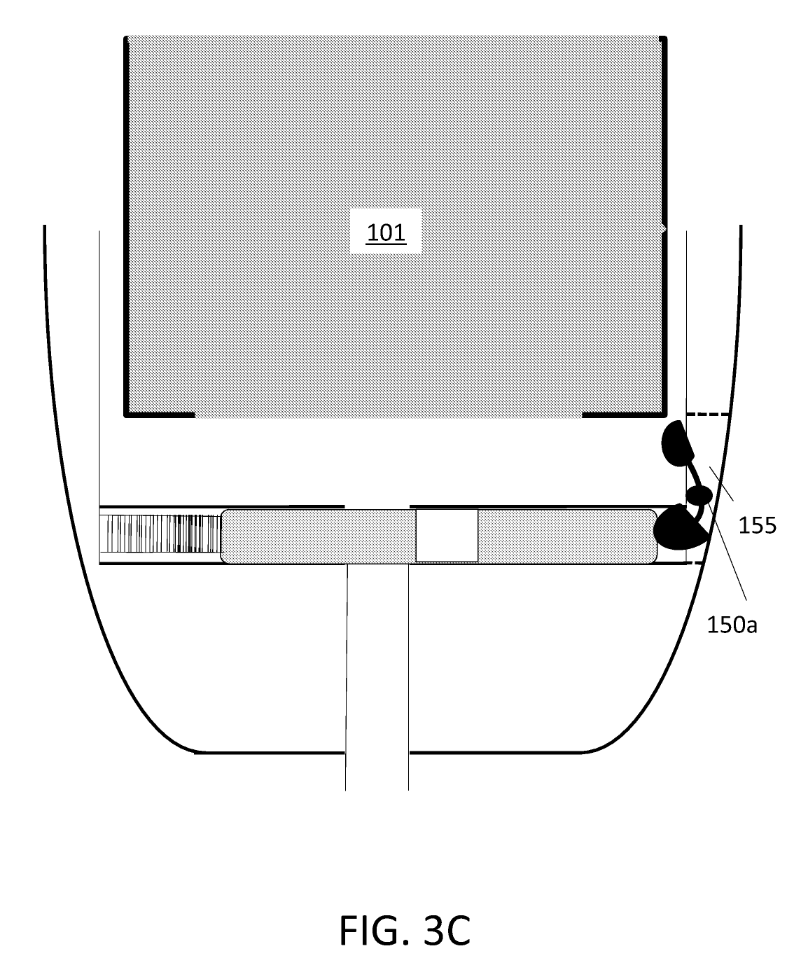

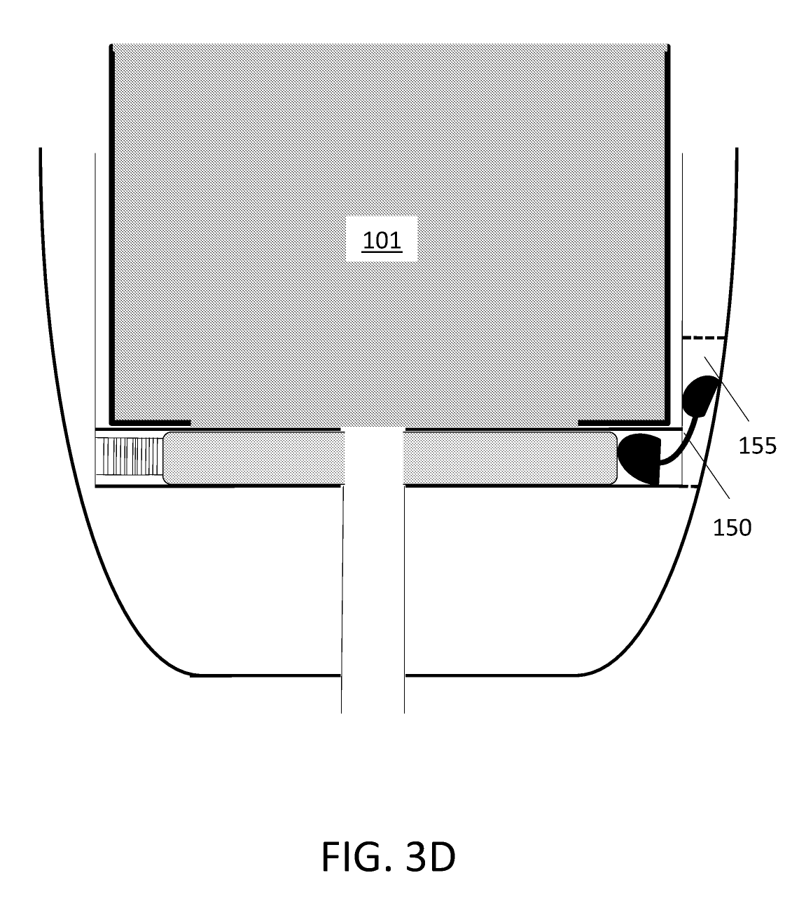

Referring to FIG. 3A-3D, in some embodiments, the resetting mechanism comprises a pivot lever (150). The pivot lever (150) may function to push back the anti-leak component (130) against the force of the biasing mechanism, e.g., the spring (140). The pivot lever (150) may be positioned in the side wall (112) of the hub (110), e.g., in a pivot lever cavity (155) disposed within the side wall (112) or directly in the side wall (112) if the side wall (112) is hollow or partially hollow. The hub (110), e.g., the side wall (112) and/or the pivot lever cavity (155) may provide for enough room to adequately accommodate the lever (150) and movement (e.g., pivoting) of the lever (150). In some embodiments, the pivot lever (150) is in contact with the chamber (120). A portion of the pivot lever (150) may protrude into the chamber (120) and optionally and/or temporarily contact the second end (132) of the anti-leak component (130). The pivot lever (150) is pivotally attached to a pivot point (150a) disposed in the hub (110) and the pivot lever (150) can pivot about the pivot point (150a). In some embodiments, the pivot lever (150) is housed in a secondary chamber within the hub (110), e.g., in the side wall (112) of the hub (110).

As shown in FIG. 3A-3D, the pivot lever (150) comprises a first end (151) and a second end (152). The first end (151) is adapted to engage the inner cavity (113) of the hub (110) via an entrance (158), e.g., a membrane disposed in the wall (113b) of the inner cavity (113) or via a hole in the wall (113b) of the inner cavity (113). The second end (152) is adapted to engage the second end (132) of the anti-leak component (130). The pivot lever (150) can pivot between a first position and a second position about the pivot point (150a). In the first position, as shown in FIG. 3A and FIG. 3C, the first end (151) is at least partially pressed into the inner cavity (113) of the hub (110) via the entrance (158). In the second position, as shown in FIG. 3B and FIG. 3D, the second end (152) of the pivot lever (150) presses against the second end (132) of the anti-leak component (130) thereby moving the anti-leak component (130) to the open position. In some embodiments, insertion of a secondary device (101) (e.g., IV tubing component) may cause the pivoting of the pivot lever (150) to the second position. In some embodiments, a practitioner can manually move the pivot lever (150) to the second position.

The pivot lever (150) is biased in the first position caused by the biasing mechanism, e.g., the spring (140) biasing the anti-leak component (130) to the closed position. In some embodiments, the presence of a secondary device (101) keeps the pivot lever (150) in the second position. In some embodiments, a locking system keeps the pivot lever (150) in the second position.

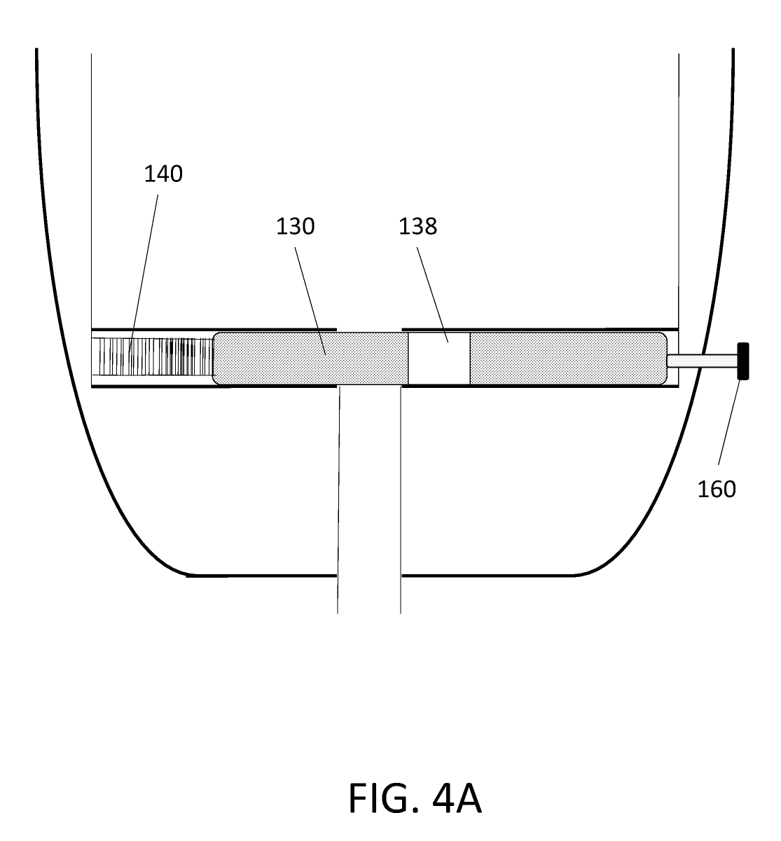

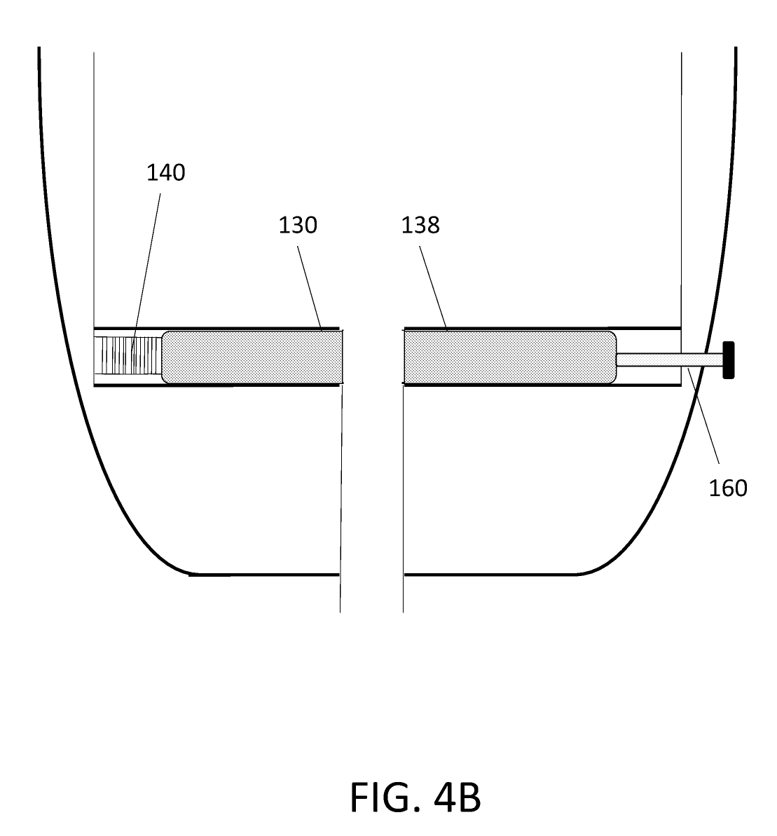

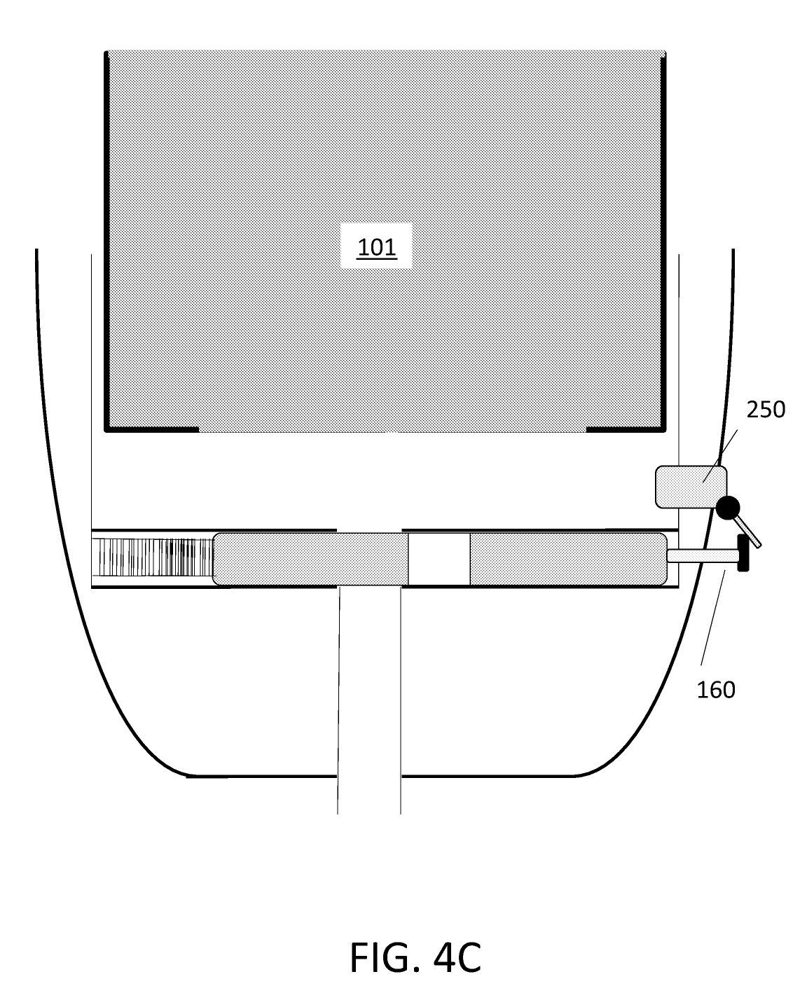

Referring to FIG. 4A-4D, in some embodiments, the resetting mechanism comprises a button (160). The button (160) may function to push back the anti-leak component (130) against the force of the biasing mechanism, e.g., the spring (140). In some embodiments, the button (160) is disposed on an outer surface of the hub (110). The button (160) traverses the hub (110) and can temporarily engage the second end (132) of the anti-leak component (130).

In some embodiments, the button (160) can move between a pressed position and an unpressed position. In the unpressed position, as shown in FIG. 4A and FIG. 4C, the button (160) does not press against the anti-leak component (130) to the point that the anti-leak component (130) moves to the open position. In some embodiments, the button (160) does not engage or touch the anti-leak component (130) when the button is in the unpressed position. In some embodiments, the button (160) engages or touches the anti-leak component (130) but does not move the anti-leak component (130) to the open position. In the pressed position, as shown in FIG. 4B and FIG. 4D, the button (160) presses against the second end (132) of the anti-leak leak component (130) to the point that the anti-leak component (130) is moved to the open position.

The button (160) may be biased in the unpressed position, e.g., caused by the biasing mechanism, e.g., the spring (140) biasing the anti-leak component (130) to the closed position. In some embodiments, the button (160) is manually pressed (e.g., moved to the pressed position), e.g., via a practitioner.

In some embodiments, the button (160) can be secured in the pressed position via a locking system. For example, as shown in FIG. 4A-D, in some embodiments, a locking system (250) is disposed on the outer surface of the hub (110), wherein the locking system (250) functions to keep the anti-leak component (130) in the open position when desired. The locking system (250) must be constructed so as not to fail so that the anti-leak component (130) is not accidentally moved to the closed position, e.g., a patient can't accidentally bump and offset the button (160) causing the close of the anti-leak component (130). The example of the locking system (250) shown in the figures is merely an example and in no way limits the present invention. The example shown in the figures is configured to secure the button (160) in the pressed position when the secondary device (101) is inserted (and properly placed) into the hub (110). In some embodiments, removal of the secondary device (101) causes the locking system (250) to allow the button to move to the unpressed position. The locking system (250) is not limited to the described and shown embodiments, and may be any appropriate locking device.

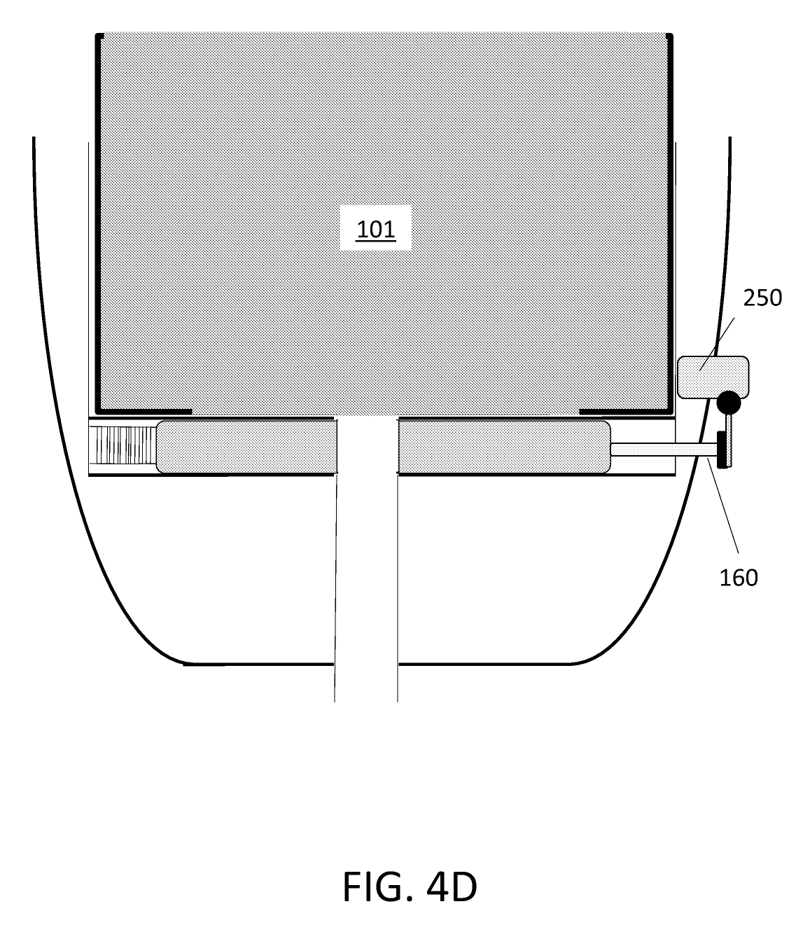

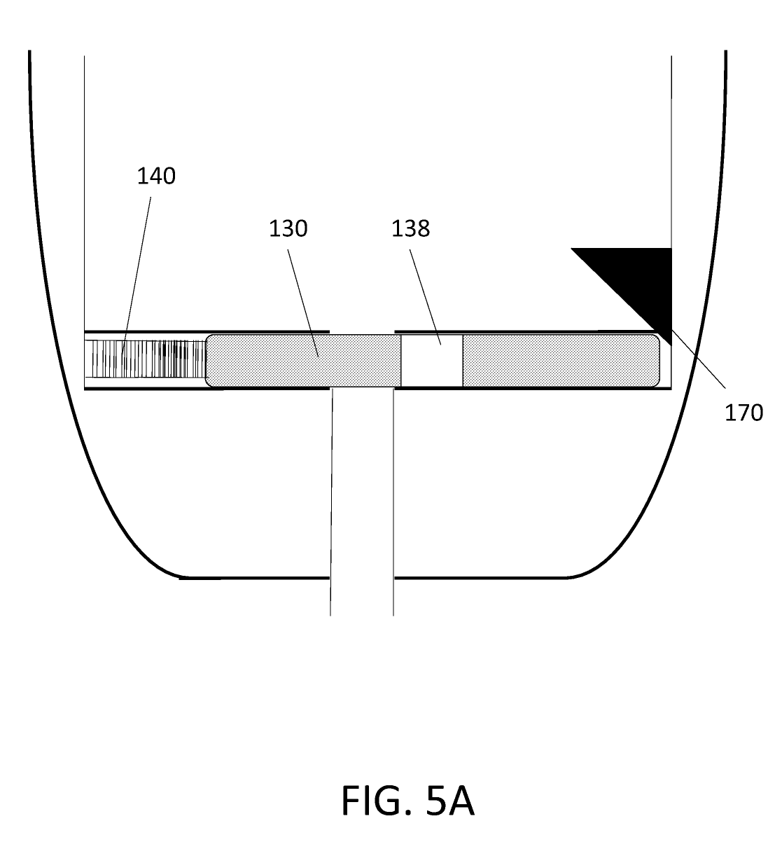

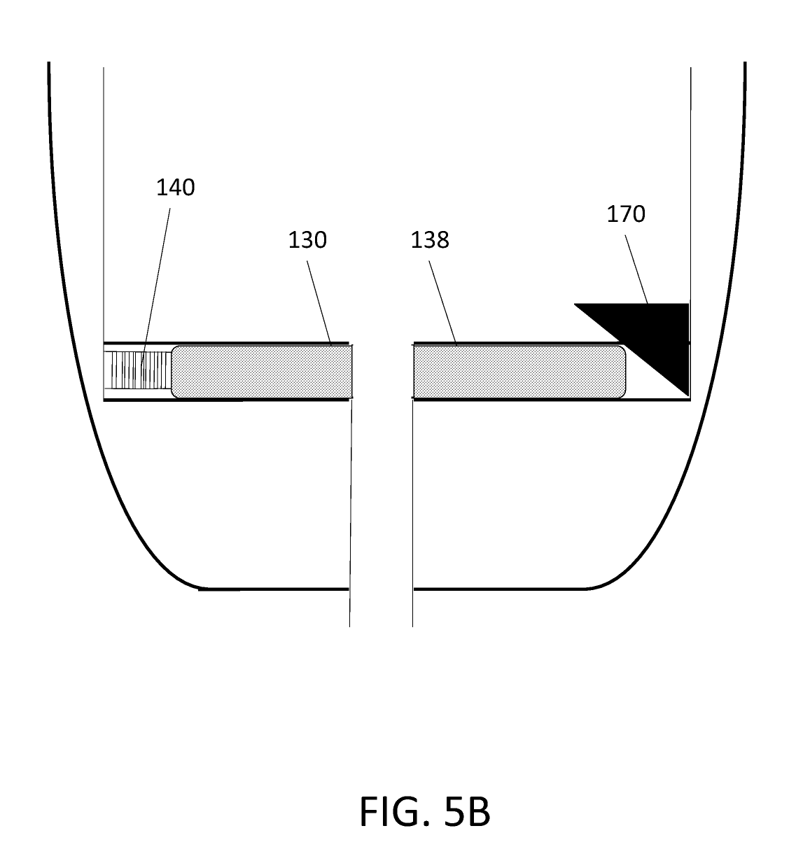

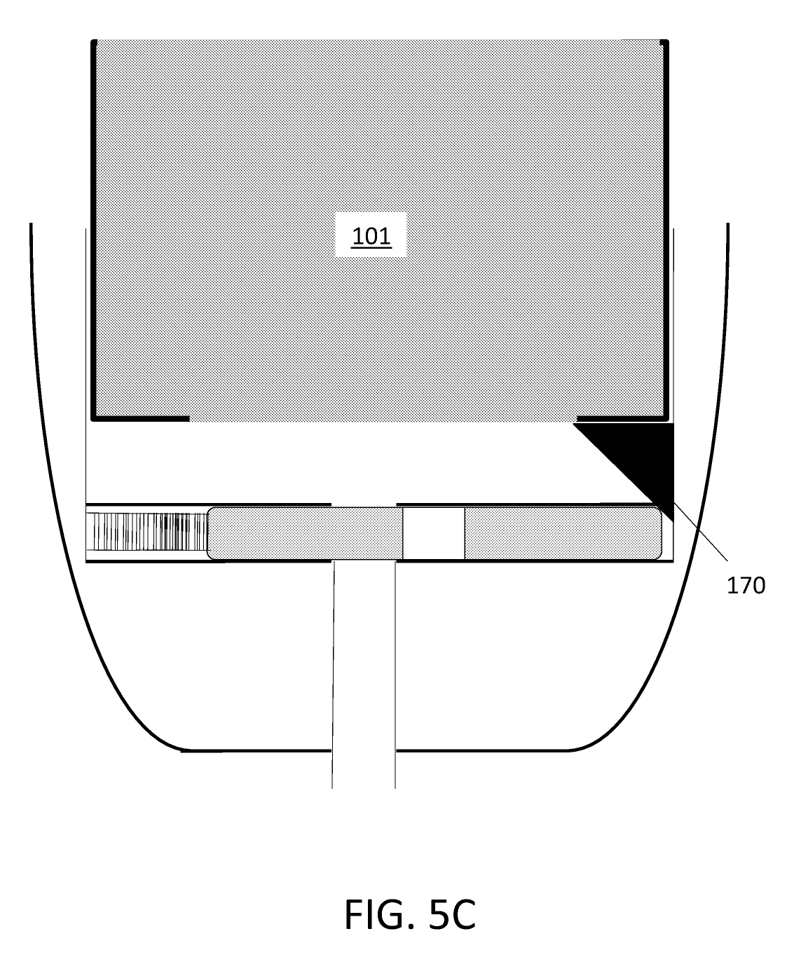

Referring to FIG. 5A-5D, in some embodiments, the resetting mechanism comprises a wedge (170). The wedge (170) is not limited to a wedge shape but may be any appropriate shape. FIG. 5A-5D show various sizes and shapes of wedges and show that the wedge (170) may optionally be compressible. The wedge (170) may function to push back the anti-leak component (130) against the force of the biasing mechanism, e.g., the spring (140). In some embodiments, the wedge (170) is disposed at least partially within the inner cavity (113) of the hub (110) and partially within the chamber (120) as shown in FIG. 5A-5D. The wedge (170) traverses the bottom surface (113a) of the inner cavity (113) of the hub (110) and can temporarily engage the second end (132) of the anti-leak component (130). In some embodiments, the wedge (170) is slidably attached to the wall (113b) of the inner cavity 113). In some embodiments, the wedge (170) is slidably or fixedly attached to a side wall of the chamber (120). In some embodiments the wedge (170) is compressible.

The wedge (170) has a pointed end, a flat end, and a hypotenuse end. The pointed end is disposed in the chamber (120). The hypotenuse end contacts or is adapted to contact the second end (132) of the anti-leak component (130). The flat end can extend upwardly through the bottom surface (113a) of the inner cavity (113) of the hub (110).

In some embodiments, the wedge (170) can move between a first position and a second position. In the first position, as shown in FIG. 5A and FIG. 5C, the wedge (170) is positioned upwardly in the inner cavity (113) of the hub (110), e.g., via the pressure of the biasing mechanism and the anti-leak component (130). In the second position, as shown in FIG. 5B and FIG. 5D, the wedge (170) is pushed downwardly into the chamber (120) and the hypotenuse end of the wedge (170) presses against the second end (132) of the anti-leak component (130) to the point that the anti-leak component (130) moves to the open position.

The wedge (170) may be biased in the first position, e.g., caused by the biasing mechanism, e.g., the spring (140) biasing the anti-leak component (130) to the closed position. In some embodiments, insertion of a secondary device (101) (e.g., IV tubing component) may cause the movement of the wedge (170) to the second position. In some embodiments, a practitioner can manually move the wedge (170) to the second position. In some embodiments, the presence of the secondary device (101) keeps the wedge (170) in the first position. In some embodiments, the wedge (170) is secured in the second position via a locking system.

System with Expandable Inner Cavity

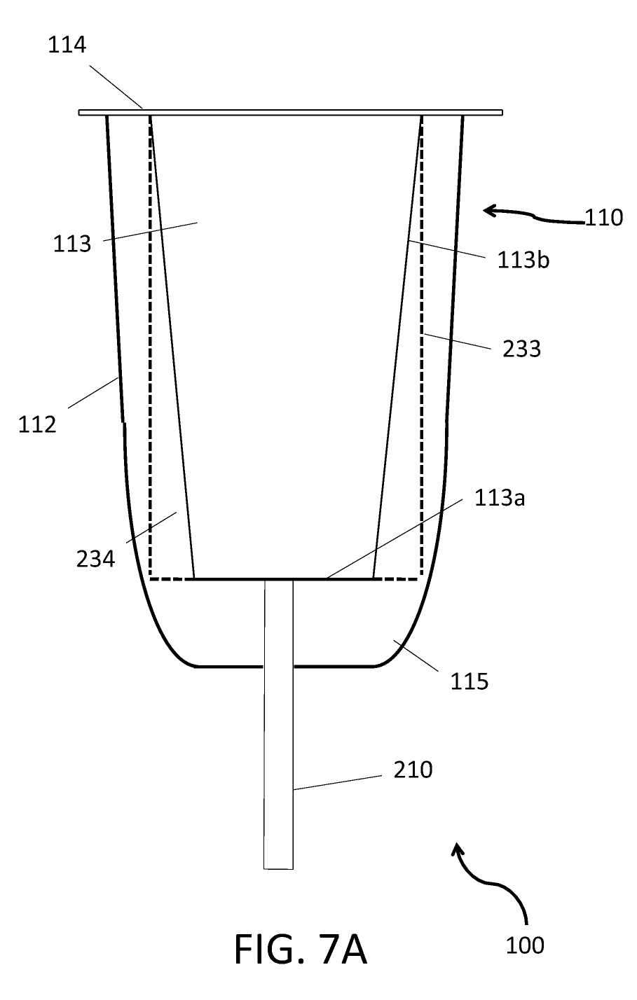

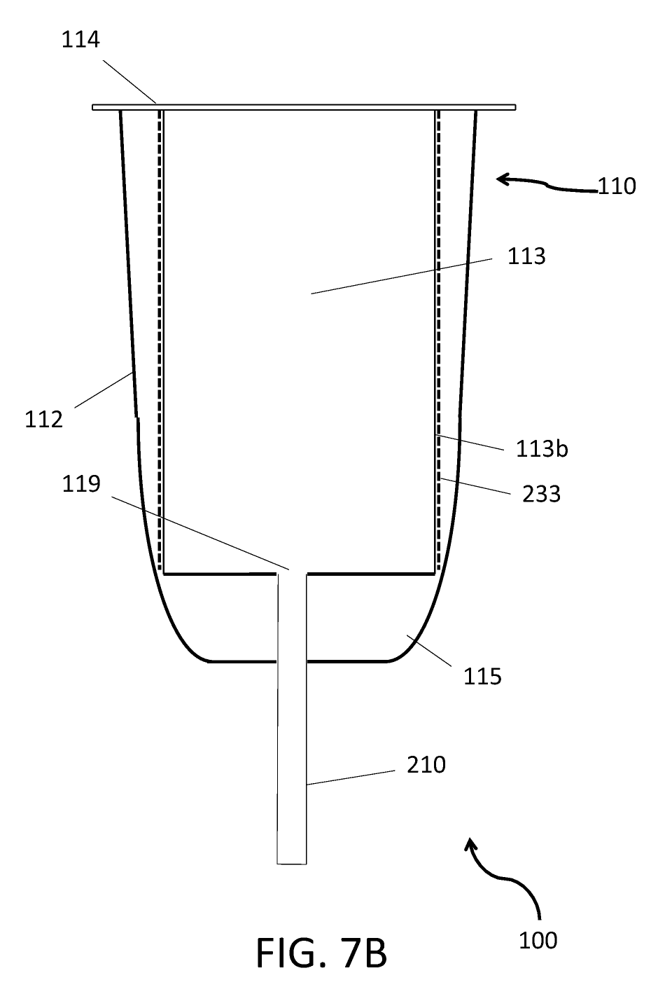

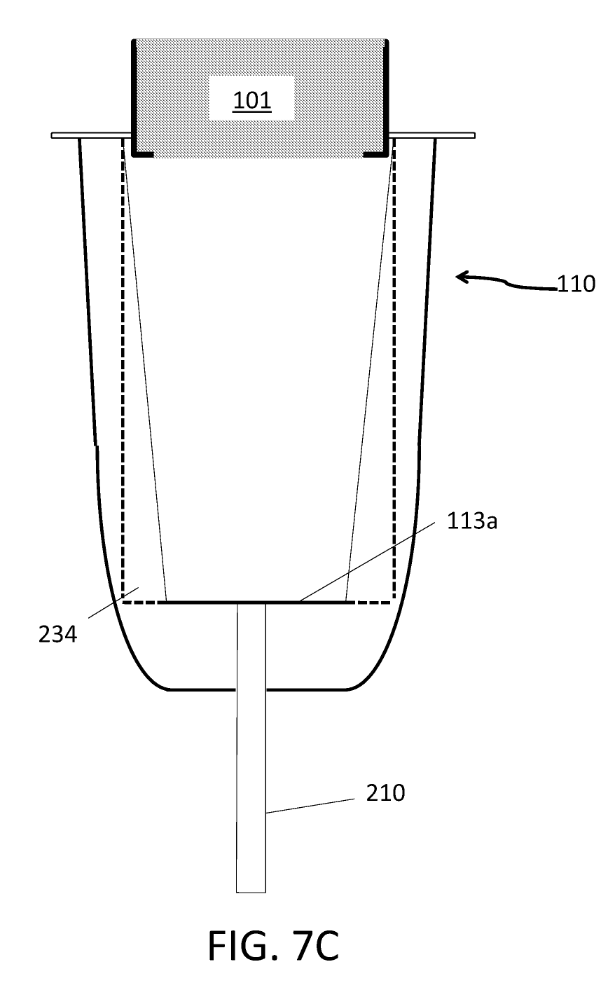

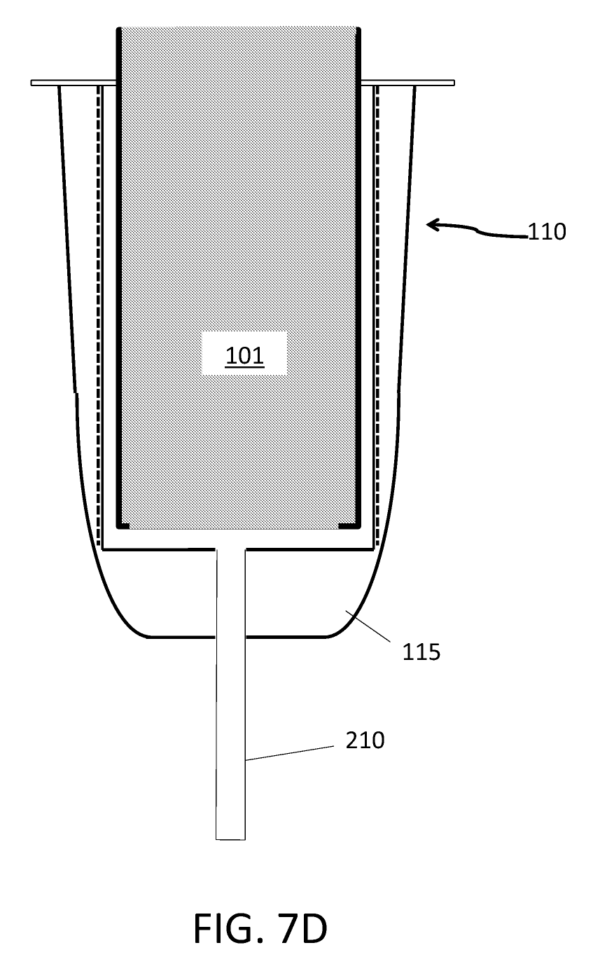

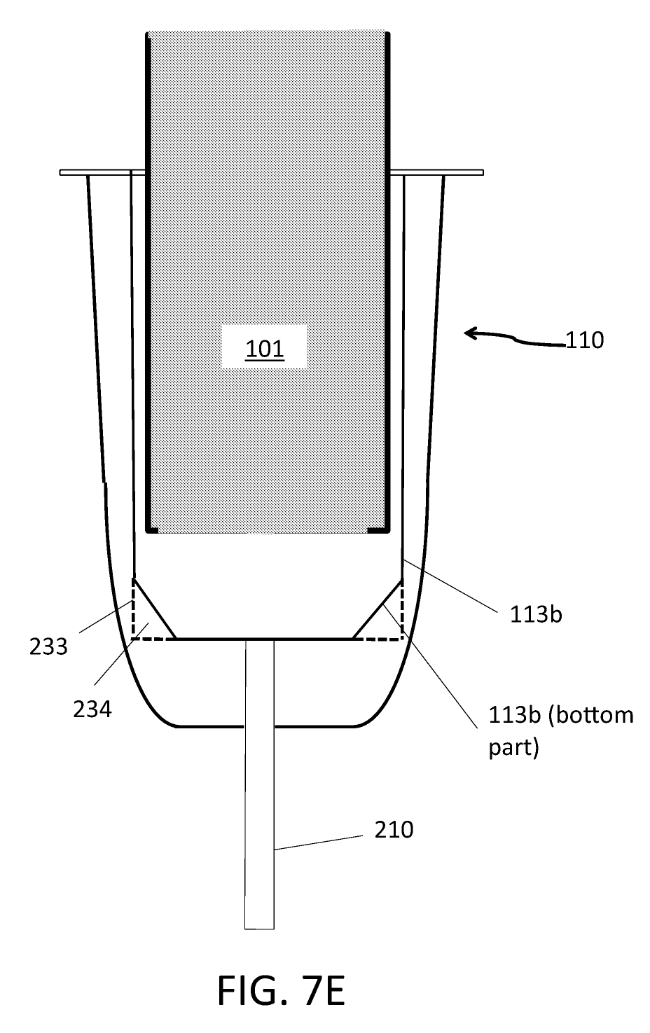

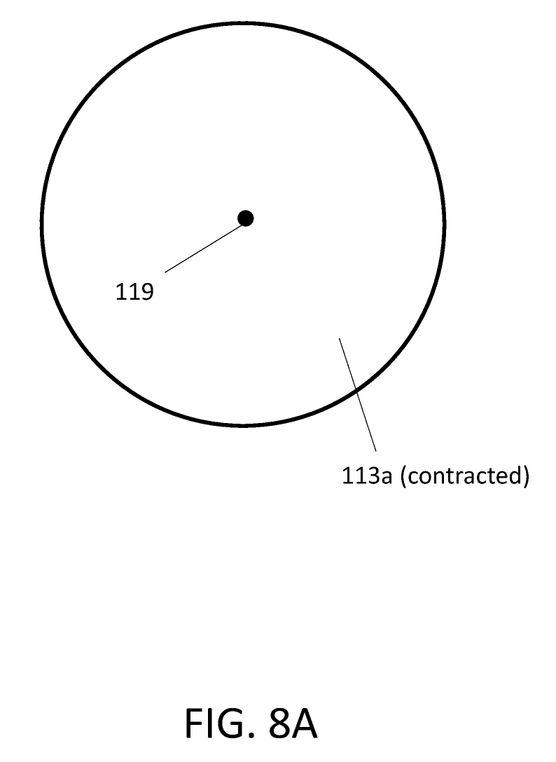

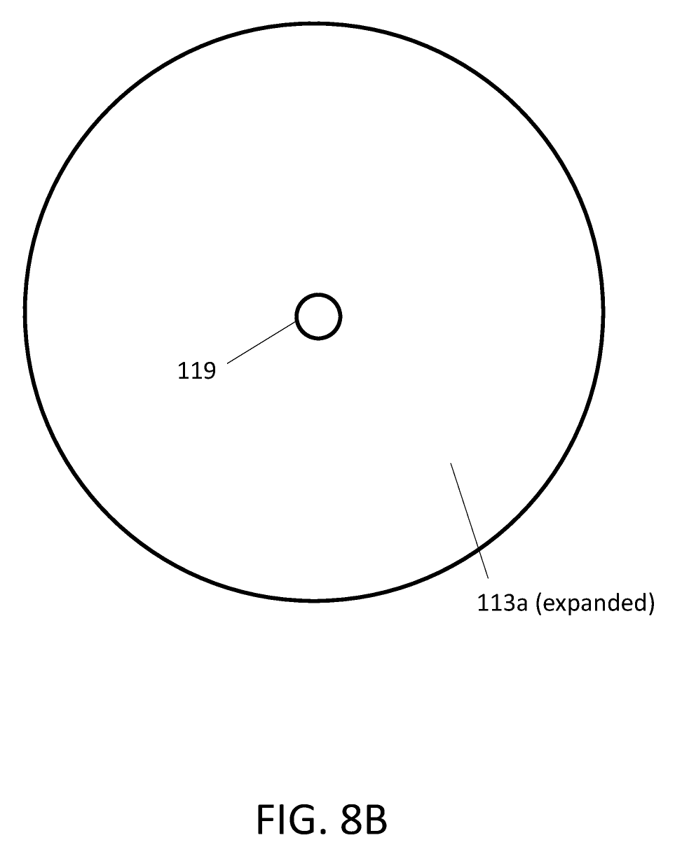

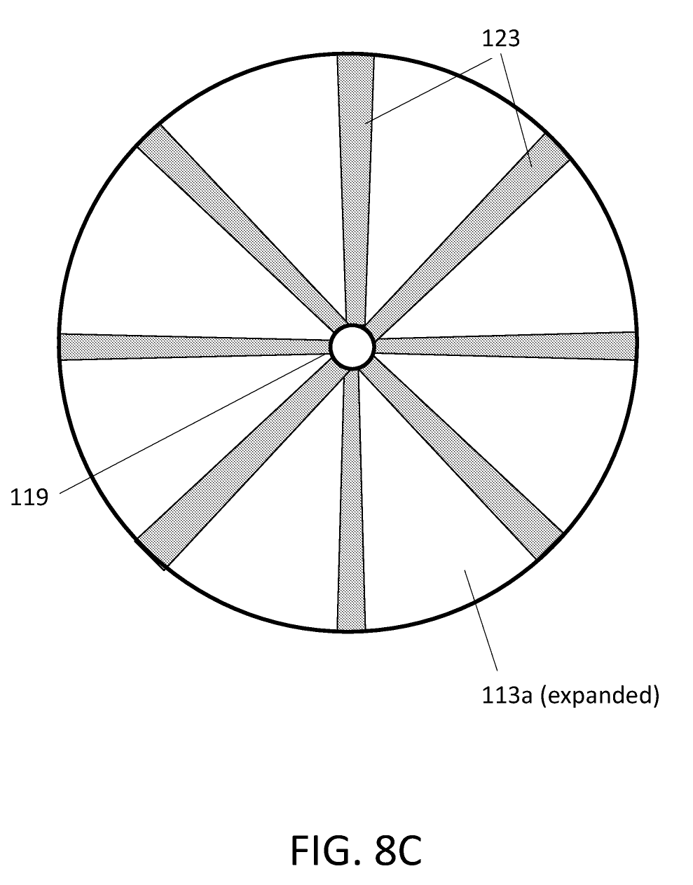

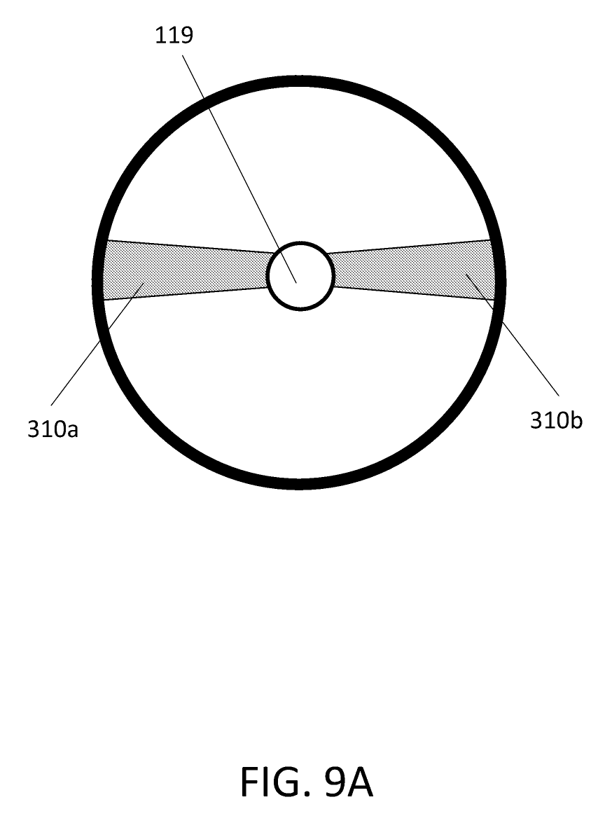

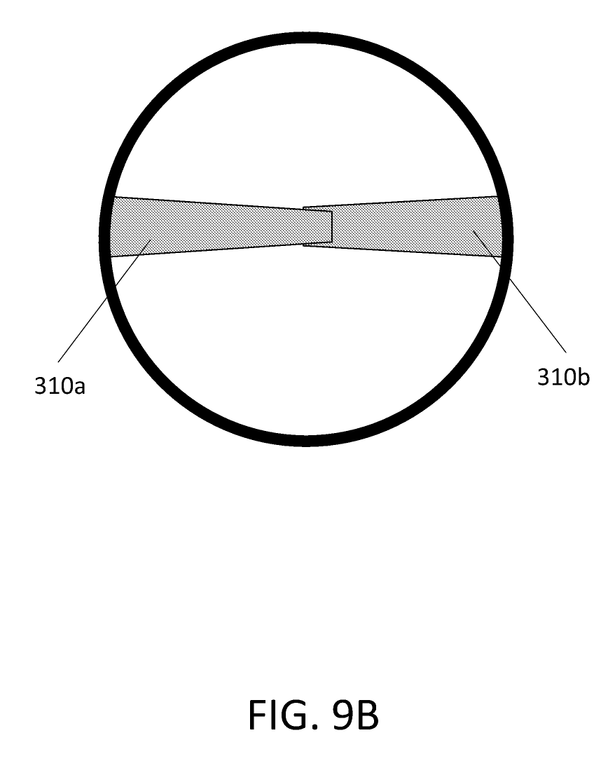

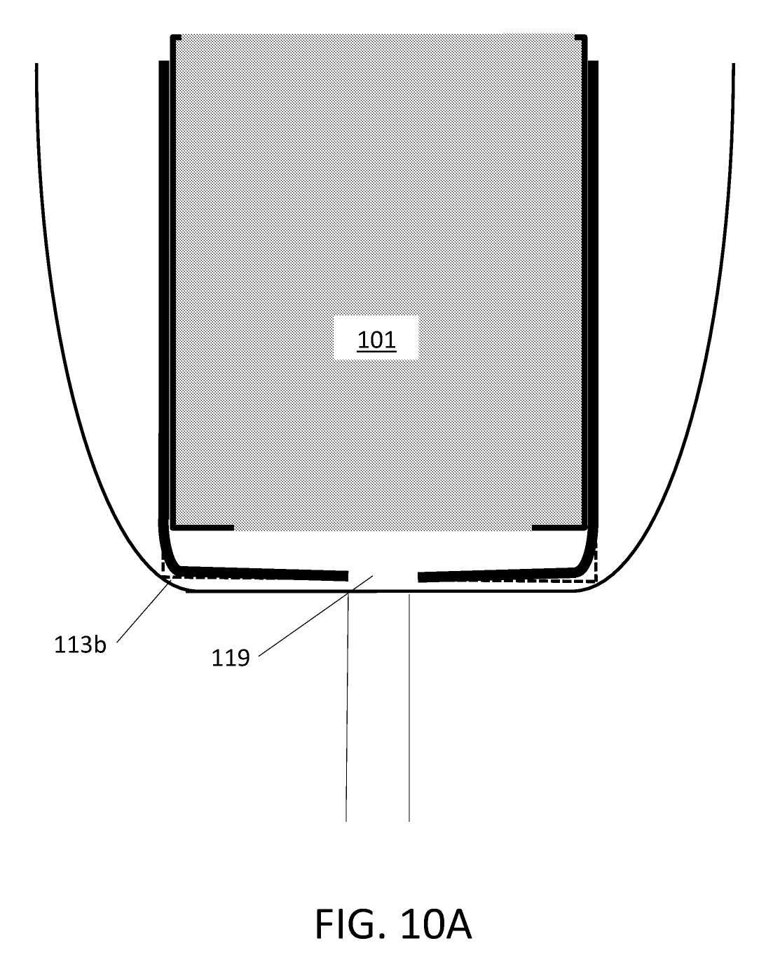

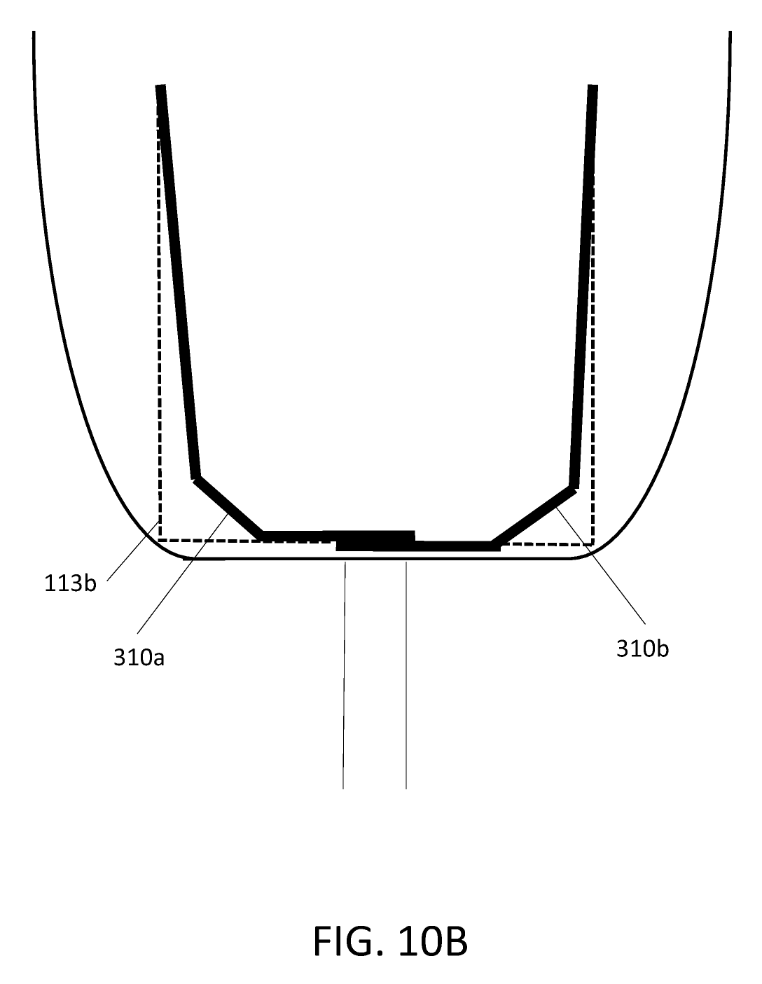

Referring now to FIG. 7A-E, FIG. 8A-C, FIG. 9A-B, and FIG. 10A-B, the present invention also features an angiocatheter system (100) comprising a hub (110) and a catheter that extends from the hub (110), wherein either the hub (110) or inner cavity (113), or components disposed in the hub (110) or inner cavity (113), regulate the opening and closing of a hole (119) in the bottom surface (113a) of the inner cavity (113) respectively allowing and preventing flow to and from the catheter (210).

For example, the system (100) of the present invention comprises a hub (110), which is a receiving end (a female receptacle) for a secondary device (101), e.g., an intravenous tubing component such as an IV line, a syringe, etc. The hub (110) has a side wall (112), an inner cavity (113), an open top end (114), and a bottom portion (115). The inner cavity (113) is accessible via the open top end (114), e.g., the secondary device (101) (IV tubing component) can be inserted into the inner cavity (113) via the open top end (114).

The inner cavity (113) is adapted to accept a secondary device (101), e.g., IV tubing component. The secondary device (101) may feature a Luer system such as a Luer lock or a Luer Slip system. Luer systems are well known to one of ordinary skill in the art and are commonly found on secondary devices. In some embodiments, the top end (114) of the hub (110) engages the Luer system of the secondary device.

A first hole (119) is disposed in a bottom surface (113a) of the inner cavity (113). The first hole (119) may be centered in the bottom surface (113a) of the inner cavity (113). The hole (119) is adapted to accept a needle (103), e.g., a needle (103) that temporarily resides in the catheter (210) that extends from the hub (110). The catheter (210) extends from the hole (119) through the bottom portion (115) of the hub (110) a distance past the bottom portion (115) of the hub (110). In some embodiments, the system (100) comprises a needle (103) temporarily sheathed by the catheter (210).

In some embodiments, the hub (110), or a portion thereof, is expandable and contractable. In some embodiments, the inner cavity (113), or a portion thereof, is expandable and contractable. The expansion of the hub (110) or inner cavity (113), or a portion thereof, may cause the opening of the hole (119). The contraction of the hub (110) or inner cavity (113), or a portion thereof, may cause the closing of the hole (119). In some embodiments, the bottom surface (113a) of the inner cavity (113) is expandable and contractable, e.g., like a membrane, wherein expansion and contraction regulates opening and closing of the hole (119). In some embodiments, the bottom surface (113a) (e.g., membrane-like structure) of the inner cavity (113) comprises ribs (123), e.g., for structural support, for elasticity, etc.

For example, in some embodiments, the bottom surface (113a) of the inner cavity (113) is expandable and when the inner cavity (113), or a portion thereof, is expanded. FIG. 7A and FIG. 7C show an example of an inner cavity (113) that is slanted or angled inwardly, e.g., the inner cavity (113) is in a contracted state (e.g., a “second position”) and the hole (119) is closed (see also FIG. 8A). As shown in FIG. 7A and 7C, the wall (113b) of the inner cavity (113) can slide inwardly and outwardly within a wall cavity (234) with respect to a secondary wall (233). FIG. 7E shows an alternative embodiment wherein only a portion of the inner cavity (113), e.g., the bottom portion, is angled and in a contracted state. When the inner cavity (113) is expanded (e.g., to a “first position”) as shown in FIG. 7B and FIG. 7D, the bottom surface (113b) of the inner cavity (113) also expands, thereby opening the hole (119) (see also FIG. 8B). In some embodiments, insertion of a secondary device (101), e.g., an IV tubing component (101) causes expansion of the inner cavity (113) and subsequent opening of the hole (119). In the open position, the hole (119) allows flow between the catheter (210) and the inner cavity (113) and/or secondary device (101). In some embodiments, removal of a secondary device (101) causes contraction of the inner cavity (113) and bottom surface (113a) of the inner cavity (113) and closing of the hole (119). The inner cavity (113) may be biased in the second position (contracted position). In some embodiments, the bottom surface (113a) of the inner cavity (113) is biased in the contracted position (second position). In some embodiments, the bias of the bottom surface (113b) of the inner cavity (113) to the contracted position biases the inner cavity (113) to the contracted position (second position).

The inner cavity (113), e.g., the wall (113b) of the inner cavity (113), and/or bottom surface (113a) of the inner cavity (113) may be constructed from any appropriate material for providing expandability and contractibility. For example, in some embodiments, the inner cavity (113), e.g., the wall (113b) of the inner cavity (113), and/or bottom surface (113a) of the inner cavity (113) is constructed from a material comprising rubber, elastic, latex, the like, or a combination thereof.

In some embodiments, the inner cavity (113), e.g., the wall (113b) of the inner cavity (113), is constructed from a plurality of concave blades (113c), e.g., concave trapezoidal blades, that are arranged to form a cylindrical shape or a cone shape. In some embodiments, a flexible membrane connects the blades together. The concave blades (113c) have a narrow end (116c) and a wide end (116d), and the narrow ends (116c) together form the first hole (119) and the bottom surface (113a) of the inner cavity (113). When the blades (113c) are pulled apart, the hole (119) is opened. When the blades (113c) are pushed together, the hole (119) closes.

Referring now to FIG. 9A-B and FIG. 10A-B, in some embodiments, flanges (310) are disposed on the wall (113b) of the inner cavity (113), and the flanges (310) regulate the opening and closing of the inner cavity (113) and hole (119). For example, in some embodiments, a first flange (310a) is disposed on the wall (113b) inner cavity (113), and an opposing second flange (310b) is disposed on the wall (113b) of the inner cavity (113). The flanges (310) may be angled. The flanges (310) extend from the wall (113b) of the inner cavity (113) into the inner cavity (113). Each flange (310) has a wide end and a narrow end, the narrow end facing toward the open top end (114) of the hub (110).

The flanges (310) can slide inwardly to a first position (“closed position”), as shown in FIG. 9B and FIG. 10B, and outwardly to a second position (“open position”), as shown in FIG. 9A and FIG. 10A, with respect to the inner cavity (113). When the flanges (310) are in the first position, the hole (119) is closed. When the flanges (310) are in the second position, the hole (119) is open.

As shown in FIG. 9B, in the first position (“closed position”), the outer edges of the wide ends of the flanges (310) overlap and cover the hole (119). As shown in FIG. 9A, in the second position (“open position”), the wide ends of the flanges (310) slide away from each other and allow access to the hole (119).

In some embodiments, the wide ends of the flanges form a portion of the bottom surface (113a) of the inner cavity (113). In some embodiments, a semicircle is disposed on the outer edge of both wide ends of the flanges (310), and the semicircles form a hole when the flanges (310) are in the second position (“open position”).

System with Twistable Base In Inner Cavity

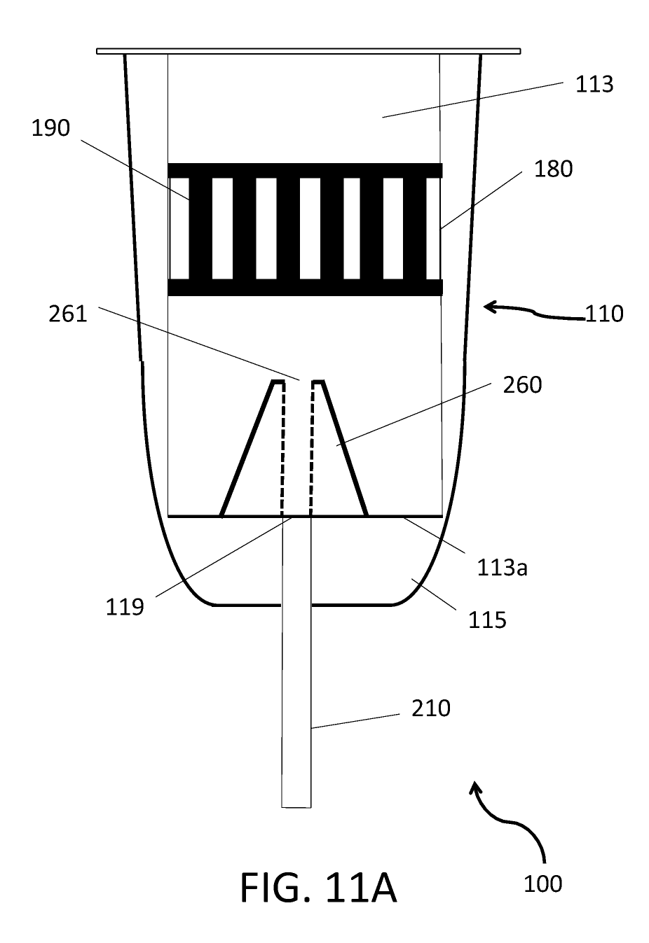

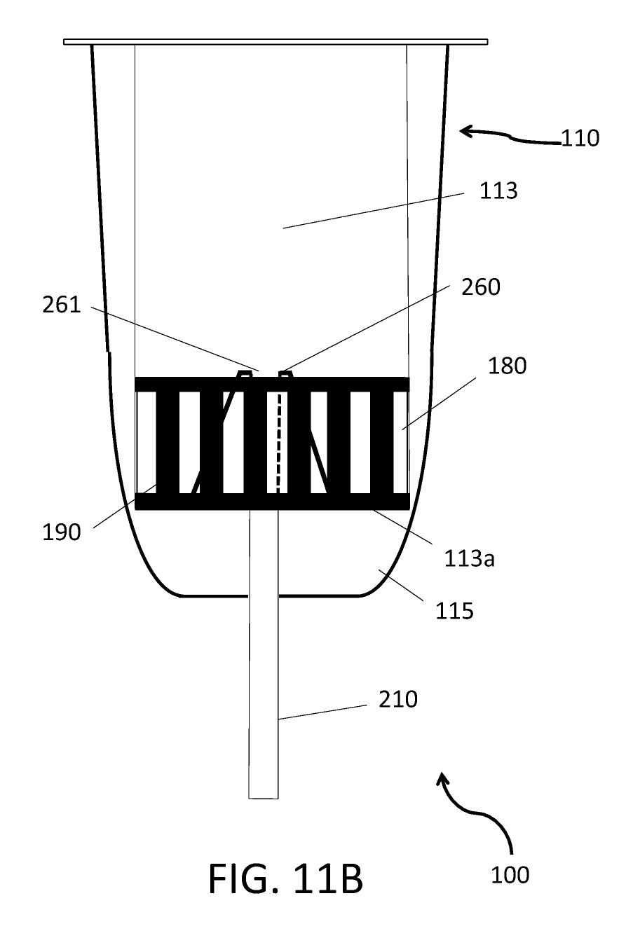

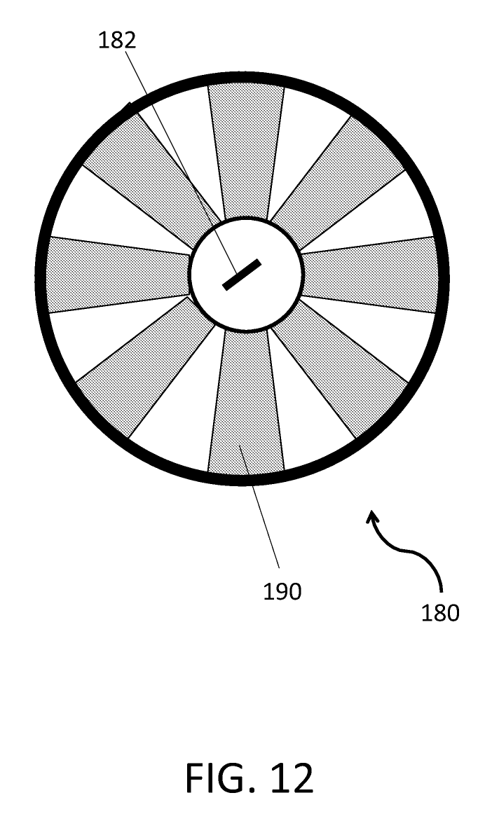

Referring now to FIG. 11A-B and FIG. 12, the present invention also features an angiocatheter system (100) comprising a hub (110), a catheter that extends from the hub (110), and a base (180) in the inner cavity (113) of the hub (110) that regulates the opening and closing of a hole (119) in the bottom surface (113a) of the inner cavity (113) respectively allowing and preventing flow to and from the catheter (210).

For example, the system (100) of the present invention comprises a hub (110), which is a receiving end (a female receptacle) for a secondary device (101), e.g., an intravenous tubing component such as an IV line, a syringe, etc. The hub (110) has a side wall (112), an inner cavity (113), an open top end (114), and a bottom portion (115). The inner cavity (113) is accessible via the open top end (114), e.g., the secondary device (101) (IV tubing component) can be inserted into the inner cavity (113) via the open top end (114).

The inner cavity (113) is adapted to accept a secondary device (101), e.g., IV tubing component. The secondary device (101) may feature a Luer system such as a Luer lock or a Luer Slip system. Luer systems are well known to one of ordinary skill in the art and are commonly found on secondary devices. In some embodiments, the top end (114) of the hub (110) engages the Luer system of the secondary device.

A first hole (119) is disposed in a bottom surface (113a) of the inner cavity (113). The first hole (119) may be centered in the bottom surface (113a) of the inner cavity (113). The hole (119) is adapted to accept a needle (103), e.g., a needle (103) that temporarily resides in the catheter (210) that extends from the hub (110). The catheter (210) extends from the hole (119) through the bottom portion (115) of the hub (110) a distance past the bottom portion (115) of the hub (110). In some embodiments, the system (100) comprises a needle (103) temporarily sheathed by the catheter (210).

In some embodiments, a base (180) is disposed in the inner cavity (113) at its bottom surface (113a). The base (180) may be cylindrical in shape. A slit (182) is disposed in the base (180) extending from its top surface to its bottom surface. The slit (182) is aligned with the hole (119) in the hub (110).

In some embodiments, the base (180) is compressible.

The base (180) can occupy an open position and a closed position respectively allowing and preventing flow from the catheter (210). In some embodiments, in the open position, the slit (182) is aligned with the hole (119) in the bottom surface (113a) of the inner cavity (113). In some embodiments, in the closed position, the slit (182) is unaligned with the hole (119) in the bottom surface (113a) of the inner cavity (113), thereby preventing flow between the catheter (210) and the inner cavity (113). In some embodiments, the base (180) can rotate, e.g., in a first direction and a second direction. In some embodiments, rotation in the first direction corresponds to moving to the open position. In some embodiments, rotation in the second direction corresponds to moving to the closed position.

As shown in FIG. 11A, in some embodiments, a cone (260) is positioned on the bottom surface (113a) of the inner cavity (113). A channel (261) extends through the cone (260), e.g., from its top surface to the bottom surface. The channel (261) is fluidly connected to the hole (119). In some embodiments, the base (180) is positioned above the cone (260). In this position, the hole (119) is closed. As shown in FIG. 11B, as a secondary device (101) is inserted into the inner cavity (113), the secondary device (101) pushes downwardly on the base (180) and the cone (260) protrudes through the slit (182), and the hole (119) effectively opens.

In some embodiments, a plurality of ribs (190) is disposed in the base (180), e.g., radially extending from the center slit (182). In some embodiments, the ribs (190) are angled (not shown in the figures). In some embodiments, the angle of the ribs (190) biases the direction of compression of the base (180) (and subsequent opening of the slit). For example, the ribs are angled from 1 degree to 89 degrees with respect to an axis down the shaft of the syringe when the syringe is coupled to the angiocatheter system, wherein the axis is the reference 0 degree. In some embodiments, the ribs are angled from about 10-45 degrees.

In some embodiments, the compressible valve need not necessarily be compressed over the cone component (as shown in the figures) to open the slit or valve. For example, insertion of a secondary device (101) causes twisting and compression of the base, not necessarily movement over the cone as shown), which can cause opening of the slit (182).

In some embodiments, the base (180) and/or the ribs (190) are constructed from a material comprising rubber, plastic, latex, elastic, the like, or a combination thereof.

Components of the system (100) of the present invention may be constructed from a variety of materials. For example, in some embodiments, one or more components constructed from a material comprising a compressible material, for example a silicone, a rubber, a thick or dense foam, a foam-like rubber material, or the like. Other materials may include plastics, polymers such as polyethylene blends, silicone co-polymers, block polymers or other elastic compressible materials that can be sterilized and are biocompatible or approved for medical use. The present invention is not limited to the aforementioned materials.

### EXAMPLES

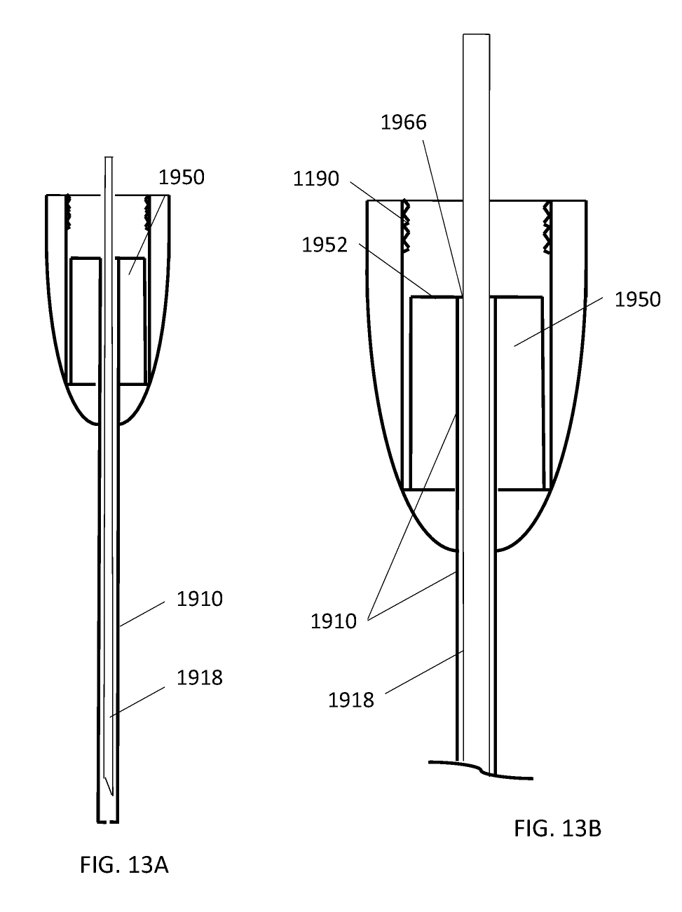

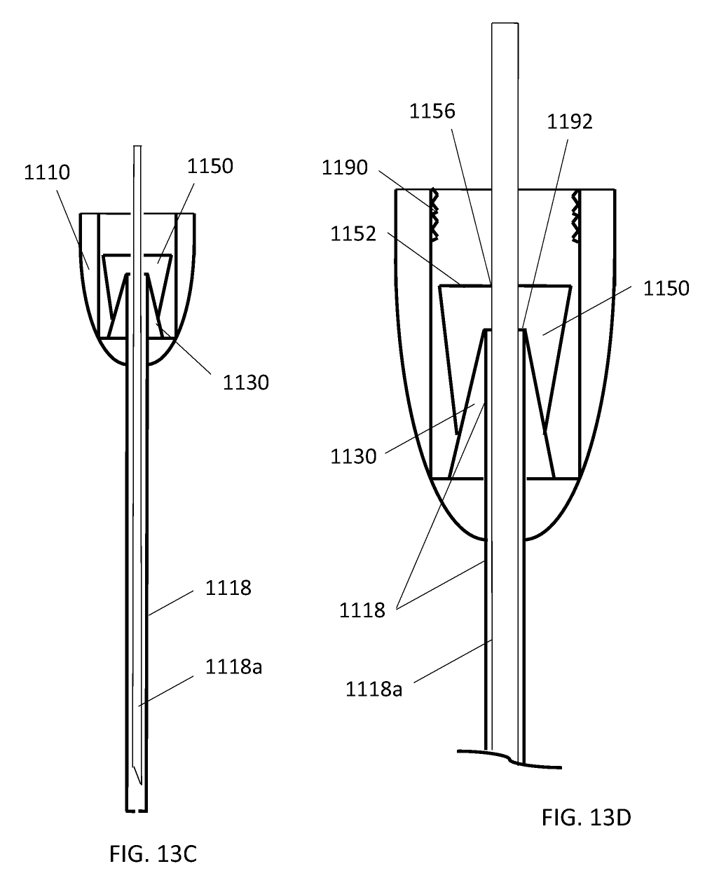

Referring to FIG. 13A-B, in some embodiments, the angiocatheter system of the present invention comprises a catheter 1910 having a distal end and a proximal end and an anti-leak component 1950 permanently disposed on the proximal end of the catheter 1910. The catheter 1910 functions as a temporary sheath for a needle 1918. In some embodiments, the anti-leak component 1950 has a receiving end (e.g., a female shaped adaptor) for receiving an end of a secondary device (e.g., a syringe, an IV line, etc.). An aperture 1956 is disposed in the receiving end and the anti-leak component 1950. The aperture 1956 allows for removal of a needle 1918. The anti-leak component 1950 provides immediate protection from leakage or blood spill. The positioning of the aperture 1965 allows for removal of the needle 1918 and immediate protection from leakage or blood spill.

The anti-leak component 1950 can move between at least an open position and a closed position respectively allowing and preventing flow through the catheter 1910. The anti-leak component 1950 is biased in the closed position and can be moved to the open position upon engagement of the secondary device with the receiving end of the anti-leak component 1950.

In some embodiments, the system further comprises the needle 1918 temporarily housed in the catheter 1910 and in the aperture 1956 of the anti-leak component 1910. The needle 1918 can be withdrawn from the catheter 1910 through the anti-leak component 1910. Removal of the needle 1918 causes the anti-leak valve 1950 to occupy the closed position to prevent flow through the cannua 1910.

The anti-leak component 1950 may be any appropriate component that allows for temporary stoppage of flow from the catheter 1910. For example, in some embodiments, the anti-leak component 1950 is a valve, e.g., a compressible valve.

FIG. 13A and FIG. 13B show the needle 1918 within the catheter 1910 and anti-leak component 1950 prior to removal. The position of the aperture 1956 in the anti-leak component 1950 allows for removal of the needle 1918 and immediate protection from leakage or blood spill.

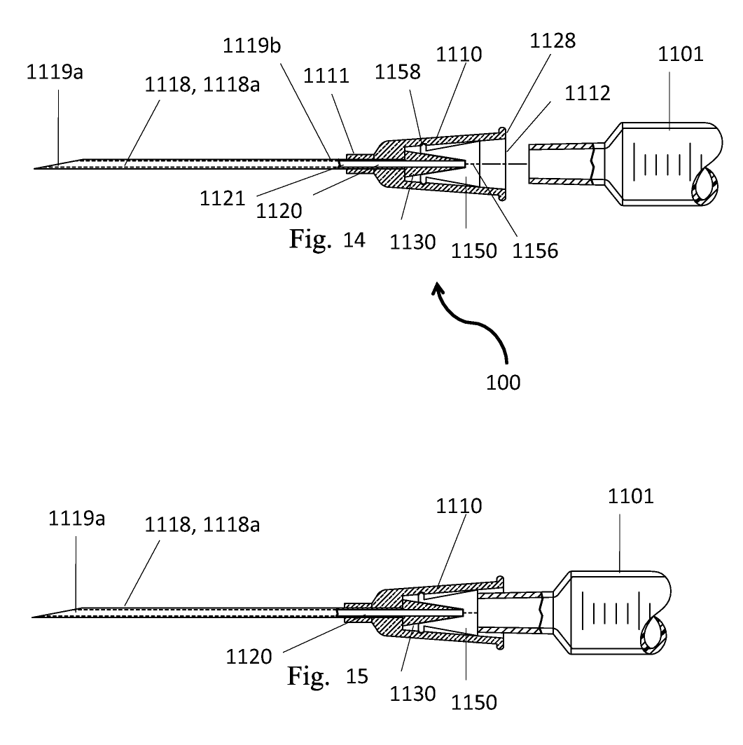

FIG. 13C, FIG. 13D, and FIG. 14-23 show more examples of systems of the present invention. The present invention is not limited to the described examples and embodiments.

Referring to FIG. 13C and FIG. 13D, the angiocatheter system of the present invention comprises a hub 1110 having a first end and a second end (the second end being open). The hub 1110 can function as an adapter for a secondary device (101) (e.g., a syringe); for example an engagement means 1190 may be disposed on or in the hub 1110 (e.g., at the second end), wherein the engagement means 1190 can engage a secondary device such as a syringe.

In some embodiments, a stationary anchor 1130 is disposed in the inner cavity of the hub 1110. The stationary anchor 1130 has a first end and a second end. In some embodiments, the stationary anchor 1130 is generally cone shaped with the second end being the end with the smaller diameter. The stationary anchor 1130 is not limited to being cone shaped, for example the stationary anchor 1130 may be generally cylindrical in shape or irregular in shape, or the like. The first end of the stationary anchor 1130 may be mounted at or near the first end of the hub 1110 (e.g., see FIG. 13, FIG. 14) and the second end of the stationary anchor 1130 faces towards the second end of the hub 1110.

The system of the present invention comprises a catheter 1118 (e.g., a sheath for a needle 1118a). The catheter 1118 functions as a temporary sheath for a needle 1118a prior to removal of the needle 1118a (the catheter 1118 remains in the patient's vessel). The catheter 1118 extends through the stationary anchor 1130 to an anchor aperture 1192 disposed in the second end of the stationary anchor 1130. The catheter 1118 extends through the first end of the hub 1110.

Mounted to the stationary anchor 1130 is an anti-leak valve (e.g., compressible valve 1150). The compressible valve 1150 (e.g., anti-leak valve) has a first end and a second end 1152. In some embodiments, the compressible valve 1150 (e.g., anti-leak valve) is generally cone shaped, wherein the first end is the end with the smaller diameter. The compressible valve 1150 (e.g., anti-leak valve) is not limited to being cone shaped, for example the compressible valve 1150 (e.g., anti-leak valve) may be generally cylindrical in shape or irregular in shape, or the like. The first end of the compressible valve 1150 (e.g., anti-leak valve) may be mounted around the stationary anchor 1130 at or near the first end or middle portion of the stationary anchor 1130. An aperture 1156 is disposed in the second end 1152 (e.g., top end) of the compressible valve 1150.

FIG. 13C and FIG. 13D show the needle 1118a within the catheter 1118 prior to removal. The anchor aperture 1192 in the second end of the stationary anchor 1130 is aligned with the aperture 1156 in the compressible valve 1150 (e.g., anti-leak valve). This positioning allows for removal of the needle 1118a and immediate protection from leakage or blood spill.

The aperture of the anti-leak valve (which allows passage of a needle 1118a) is situated on the second end of the anti-leak valve 1150 (e.g., the second end of the compressible valve). The positioning of the aperture allows for removal of the needle 1118a and immediate protection from leakage or blood spill. And, as opposed to a system wherein the adapter and the angiocatheter are separate pieces (e.g., see U.S. Pat. No. 5,700,248 to Lopez), the system of the present invention features a built-in adapter (e.g., an adapter and angiocatheter are merged together), wherein the adapted is for engaging other devices including but not limited to syringes and IV lines. The positioning of the aperture in the anti-leak valve allows for such a one-piece configuration. The one-piece configuration allows for removal of the needle and immediate stop of flow by the anti-leak valve, and an adapter does not need to be attached subsequent to removal of the needle.

Referring to FIG. 13-17, in some embodiments, the angiocatheter system comprises a hub 1110 (e.g., a generally hollow hub 1110) having a first end 1111 and a second end 1112. The first end 1111 of the hub 1110 is connected (e.g., fluidly connected) to the catheter 1118 (with the needle 1118a optionally inserted through the catheter 1118) and the second end 1112 engages a secondary device (101), e.g., a syringe, IV system, blood collection tube, or the like. Generally, the first end 1119a of the catheter 1118 (or needle 1118a) is the portion that is inserted and anchored into the patient's vessel. The second end 1119b of the catheter 1118 attaches to the first end 1111 of the hub 1110 (in some embodiments, the second end 119b of the catheter 1118 extends into the inner cavity of the hub 1110 via the first end 1111 of the hub 1110). The second end 1112 of the hub 1110 is generally open, providing access to the inner cavity of the hub 1110. The hub 1110 may be generally cone shaped or cylindrical in shape, however the hub 1110 is not limited to the aforementioned shapes. In some embodiments, a lip 1128 is disposed around the second end 1112 of the hub 1110. The lip 1128 may provide a surface with which a secondary device (101), e.g., a syringe, IV system, or blood collection tube can engage to secure the hub 1110.

In some embodiments, a stationary anchor 1130 is disposed in the inner cavity of the hub 1110. The stationary anchor 1130 has a first end and a second end 1132. In some embodiments, the stationary anchor 1130 is generally cone shaped with the second end 1132 being the end with the smaller diameter. The stationary anchor 1130 is not limited to being cone shaped, for example the stationary anchor 1130 may be generally cylindrical in shape or irregular in shape, or the like. The first end of the stationary anchor 1130 may be mounted at or near the first end 1111 of the hub 1110 (e.g., see FIG. 13, FIG. 14) and the second end 1132 of the stationary anchor 1130 faces towards the second end 1112 of the hub 1110.

In some embodiments, a channel 1120 is disposed in the stationary anchor 1130, wherein the first end 1121 of the channel 1120 is positioned at the first end 1111 of the hub 1110 and the second end 1122 of the channel 1120 is positioned at the second end 1132 of the stationary anchor 1130 (see FIG. 13, FIG. 14). The channel 1120 fluidly connects the first end 1111 of the hub 1110 to the second end 1132 of the stationary anchor 1130. In some embodiments, a catheter 1118 (and/or needle 1118a) is attached (or is insertable, removably attached, etc.) to the first end 1111 of the hub and is fluidly connected to the channel 1120. In some embodiments, a portion of the catheter 1118 (e.g., the second end 1119b) traverses the stationary anchor 1130, wherein the second end 1119b of the catheter 1118 is positioned at the second end 1132 of the stationary anchor 1130. The catheter 1118 allows fluid to pass to or from the second end 1132 of the stationary anchor 1130 through the catheter 1118 (and/or needle 1118a). For example, the catheter 1118 fluidly connects the second end 1132 of the stationary anchor 1130 to the first end 1111 of the hub 1110 (and typically the first end 1119a of the catheter 1118). The catheter 1118 typically extends beyond the first end 1111 of the hub 1110, however the system of the present invention may not necessarily be constructed with a full catheter 1118. Or, in some embodiments, a cannula or cannula extension can be attached to the first end 1111 of the hub 1110.

Mounted to the stationary anchor 1130 is a compressible valve 1150. The compressible valve 1150 has a first end and a second end 1152. In some embodiments, the compressible valve 1150 is generally cone shaped, wherein the first end is the end with the smaller diameter. The compressible valve 1150 is not limited to being cone shaped, for example the compressible valve 1150 may be generally cylindrical in shape or irregular in shape, or the like. The first end of the compressible valve 1150 may be mounted around the stationary anchor 1130 (e.g., via a mounting component 1158) at or near the first end or middle portion of the stationary anchor 1130. In some embodiments, an indentation is disposed in the first end of the compressible valve 1150 adapted to accept the second end 1132 of the stationary anchor 1130 (e.g., the compressible valve 1150 fits over the second end 1132 of the stationary anchor 1130). A slit or tiny hole 1156 is disposed in the second end 1152 of the compressible valve 1150.

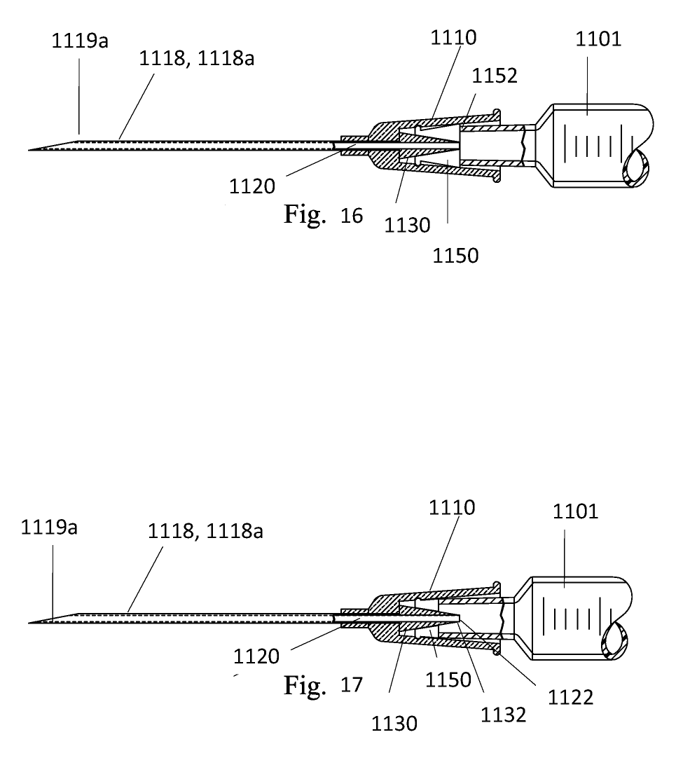

The compressible valve 1150 can be compressed, thus the compressible valve 1150 can move between multiple positions including an extended position and a compressed position, wherein the compressible valve 1150 is biased in the extended position caused by the material of the compressible valve 1150. In the extended position, as shown in FIG. 14, the second end 1152 of the compressible valve 1150 is positioned closer to the second end 1112 of the hub 1110 than is the second end 1132 of the stationary anchor 1130. The hole 1156 of the compressible valve 1150 is closed (e.g., by way of the nature of the material of the compressible valve 1150). In the extended position, the hole 1156 is closed and the compressible valve 1150 blocks access to the channel 1120, e.g., the second end 1122 of the channel 1120, (and optionally the needle 1118a and/or catheter 1118), thereby preventing blood from the vessel from leaking into the hub 1110. In the compressed position, as shown in FIG. 17, the compressible valve 1150 is compressed such that the second end 1132 of the stationary anchor 1130 is closer to the second end 1112 of the hub 1110 than is the second end 1152 of the compressible valve 1150. The second end 1132 of the stationary anchor 1130 is effectively exposed through the hole 1156 of the compressible valve 1150 (which is opened and compressed when the compressible valve 1150 is in the compressed position). In the compressed position a secondary device (101), e.g., syringe, IV system, or blood collection tube can access the channel 1120 (e.g., and optionally the needle 1118a and/or catheter 1118) via the second end 1132 of the stationary anchor 1130 for fluid transfer. FIG. 17 shows a syringe fluidly connected to the channel 1120. FIG. 15 and FIG. 16 show positions in between the extended position and the compressed position. The compressible valve 1150 may be compressed by a compressing component 5000, for example a syringe tip. In some embodiments, the compressible valve 1150 is biased in the extended position and will return to the biased extended position from the compressed position after a compressing component 5000 is no longer pressing onto the compressible valve 1150.

In some embodiments, the compressible valve 1150 is constructed from a material comprising a compressible material, for example a silicone, a rubber, a thick or dense foam, a foam-like rubber material, or the like. Other materials may include polymers such as polyethylene blends, silicone co-polymers, block polymers or other elastic compressible materials that can be sterilized and are biocompatible or approved for medical use.

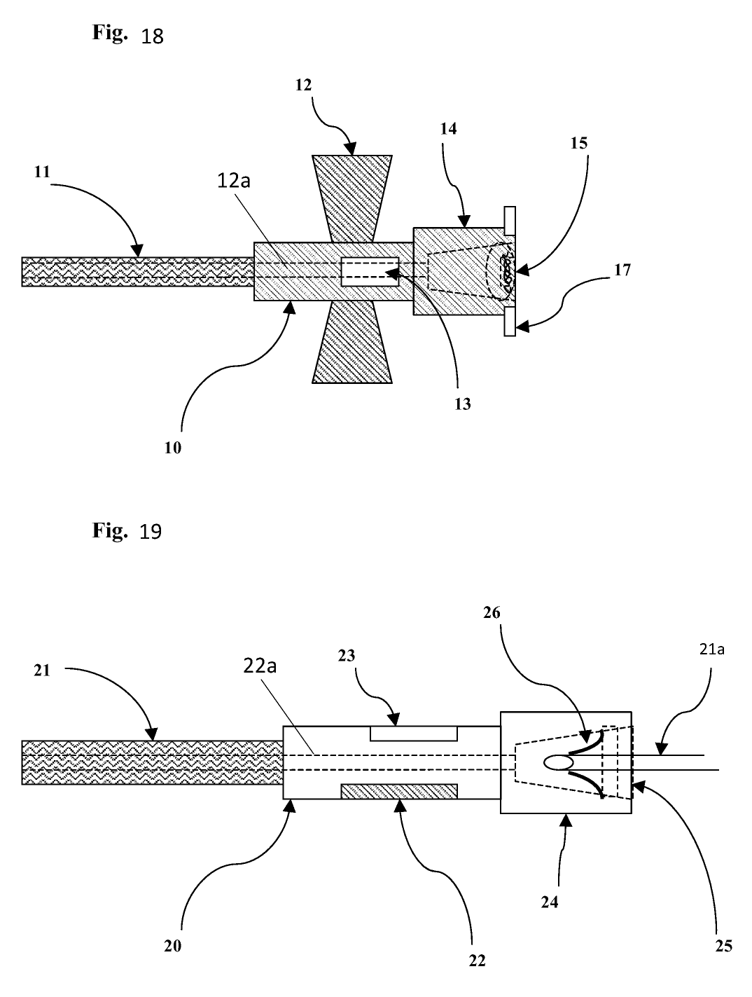

In some embodiments, a window (e.g., a blood flash window) (e.g., see FIG. 18) is disposed in the hub 1110, which allows for visualization of blood flash to confirm that the practitioner has fluidly connected the device to the vein appropriately. For example, a portion of the hub 1110 may be constructed from a translucent, transparent, or clear material. Optionally, other portions of the hub 1110 (or all of the hub 1110) may be constructed from a translucent, transparent, or clear material. As used herein, the terms “clear,” “translucent,” and “transparent” refer to a property of a material that allows visualization of light, an object, or a shadow.

In some embodiments, stabilizing tabs (e.g., see FIG. 18) are disposed on and protrude from the sides of the hub 1110. The stabilizing tabs can help prevent turning or twisting of the system once the system is inserted and secured in the patient.

Referring to FIG. 18, the angiocatheter system of the present invention comprises a hub 10 having a first end and a second end. The hub 10 allows fluid connectivity throughout the device (except when a valve is closed to stop blood flow). Disposed in the hub 10 is a blood flash window 13, which allows for visualization of blood flash to confirm that the practitioner has fluidly connected the device to the vein appropriately. The window 13 may be constructed from a clear, translucent, or transparent material. Optionally, the remaining portions of the hub 10 may be constructed from a less transparent, clear, or translucent material in order to make visual confirmation that the blood vessel has been penetrated as easy as possible for the health care provider.

In some embodiments, a channel 12a is disposed in the hub 10, which can be fluidly connected to the catheter 11. The catheter 11 (e.g., the component inserted/anchored into the patient's vessel) may be constructed from a biologically compatible plastic material. The catheter 11 may be attached to the first end of the hub (e.g., via a welding, molding or gluing process, etc.) and be fluidly connected to the channel 12a (the channel 12a extends into the inner cavity of the hub 10/hub 14).

In some embodiments, the device further comprises a connection hub 14 (having a first end and a second end 15), which may be a molded part of the hub 10 (e.g., FIG. 14-17 show the connection hub 14 as being part of the hub 10). For example, the hub 14 is a part of the hub 10 at the second end of the hub 10. The connection hub 14 is generally hollow with an open second end 15. The connection hub 14 allows for connection of the medical (e.g., IV) tubing or syringe (or other fitting such as a luer lock system). In some embodiments, connection hub tabs 17 are disposed on the outer edge of the second end of the connection hub 14 (e.g., similar to the lip 128 on the second end 1112 of the hub 1110 in FIG. 14-17). The connection hub tabs 17 may engage the medical tubing or syringe or other fitting.

In some embodiments, stabilizing tabs 12 are disposed on and protrude from the sides of the hub 10. The stabilizing tabs 12 can help prevent turning or twisting of the device once the device is inserted and secured in the patient.

Referring to FIG. 19, in some embodiments, the device comprises a reed-type valve 26 (e.g., constructed from a biocompatible plastic, optionally with fiber reinforcement). The needle 21a can be pulled out of the hub 20 and hub 24 once the device (e.g., the cannula) is inserted and anchored in a patient's vessel (e.g., the flexible cannula 21 has penetrated the vessel). In some embodiments, when the needle 21a is pulled out, the reed-type valve 26 closes tightly behind it (preventing flow of blood from the vessel into the hub 20 and hub 24). In some embodiments, a needle can be reinserted (through the reed-type valve 26) to access fluid or blood for samples or to administer a medication or other fluid, for example the needle 21 a can penetrate the opening at the second end 25 of the hub 24. Additionally shown in FIG. 18 are a flash window 23 and stabilizing wings 22. In some embodiments, a channel 22a is disposed in the hub 20 and is fluidly connected to the cannula 21.

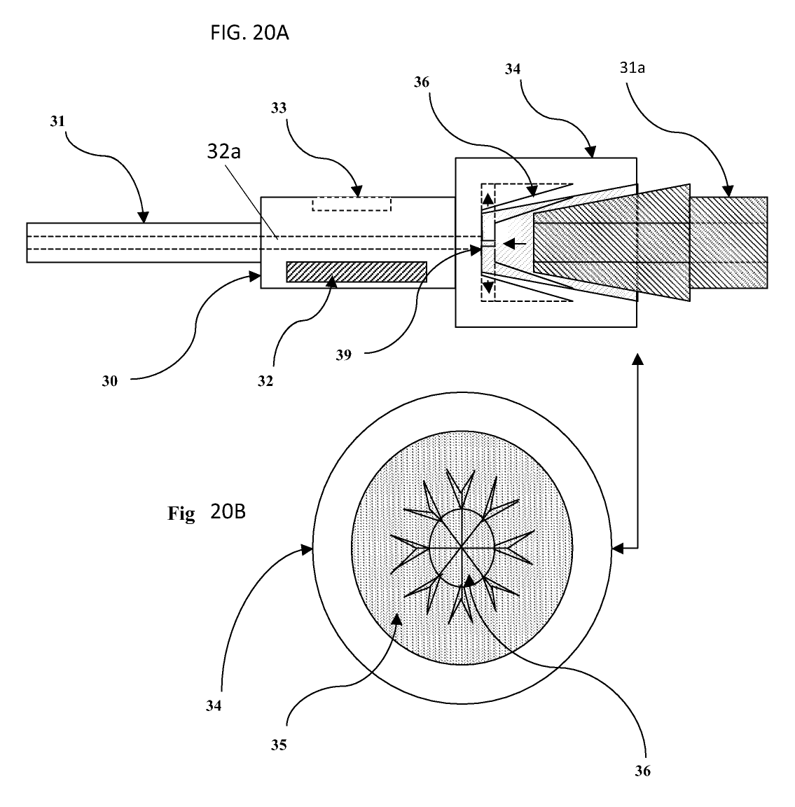

Referring to FIG. 20A and 20B, in some embodiments, the deivce comprises a pinching finger-like tines valve 36. The tines valve 36 may be constructed from a material comprising a biocompatible plastic, however the valve 36 is not limited to the aforementioned materials. In some embodiments, the needle (previously inserted into the device) holds the tines or fingers of the tines valve 36 open until the needle can be pulled out of the hub 30 and hub 34. In some embodiments, once the device is inserted/anchored in a patient's vessel (e.g., with the needle and cannula 31), the needle is pulled out and the tines valve 36 closes tightly behind it. In some embodiments, a needle may not necessarily be able to be reinserted to access fluid or blood for samples. In some embodiments, this may be accomplished by using a standard medical fitting 31 a, which when pushed in to the open second end 35 of the hub 34 pushes the fingers or tines of the tines valve 36 to the open position 39 allowing blood vessel access and fluid connection through the catheter. The tines and hub may be manufactured for example, but not limited to, by injection molding as two parts to be welded or glued together later in the assembly process. In some embodiments, the device further comprises stabilizing tabs 32 and a blood flash window 33. A window 33 is shown on the hub 30 in FIG. 20A, however a window 33 may alternatively located on the hub 34. In some embodiments, a channel 32a is disposed in the hub 30 and is fluidly connected to the cannula 31.

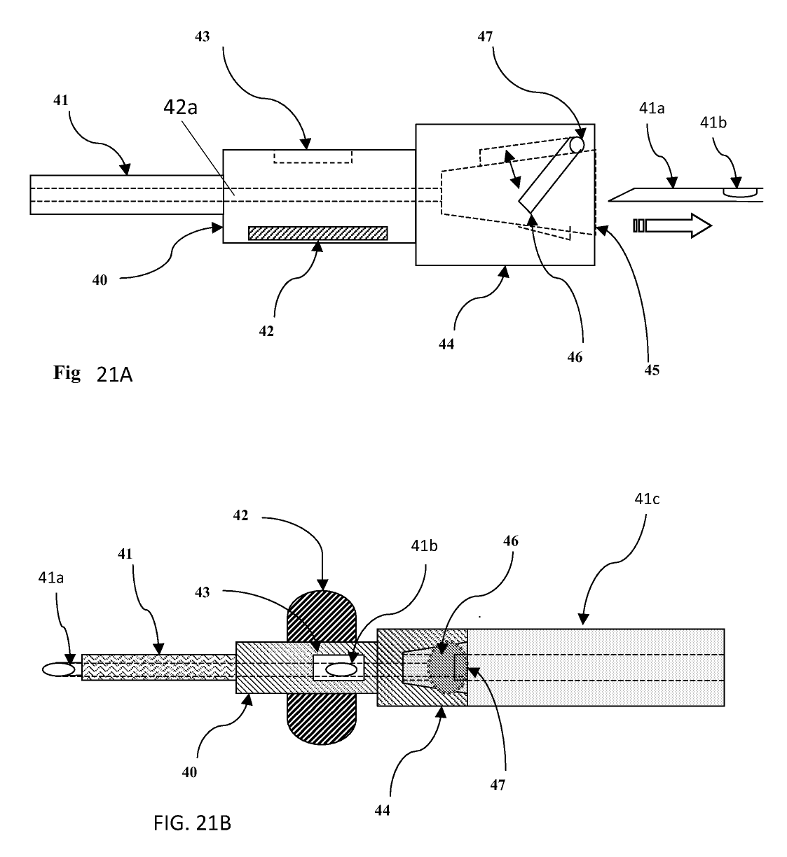

Referring to FIG. 21A, in some embodiments, the device comprises a spring-tensioned clapper-type valve device 46 that closes and seals the open second end 45 of the hub 44 as the needle 41a is pulled out of the cannula 41, hub 40, and catheter hub 44. In some embodiments, the spring 47 of the spring-tensioned clapper-type valve 46 allows the valve 46 to move to the closed position when the needle 41a is removed (e.g. the spring 47 biases the valve 46 in the closed position). Also shown in FIG. 21A is a flash window 43 in the hub 40 and a needle 41a with an opening 41b. Also shown in FIG. 21A are stabilizing tabs 42. In some embodiments, a channel 42a is disposed in the hub 40 and is fluidly connected to the cannula 41.

FIG. 21B shows the embodiment of FIG. 21A with a fully inserted needle 41a and a needle stick safety device 41c that retracts the needle 41a into its body to protect care providers from accidental needle sticks. In some embodiments, when the fully assembled the needle stick safety device 41c holds the spring-tensioned clapper-type valve 46 open in a recessed position within the catheter hub 44. In some embodiments, the needle 41a is fully extended through the hub 40 and cannula 41 with the needle opening 41b aligned with the flash window 43. The tip of the needle 41a is exposed to enable penetration of the blood vessel.

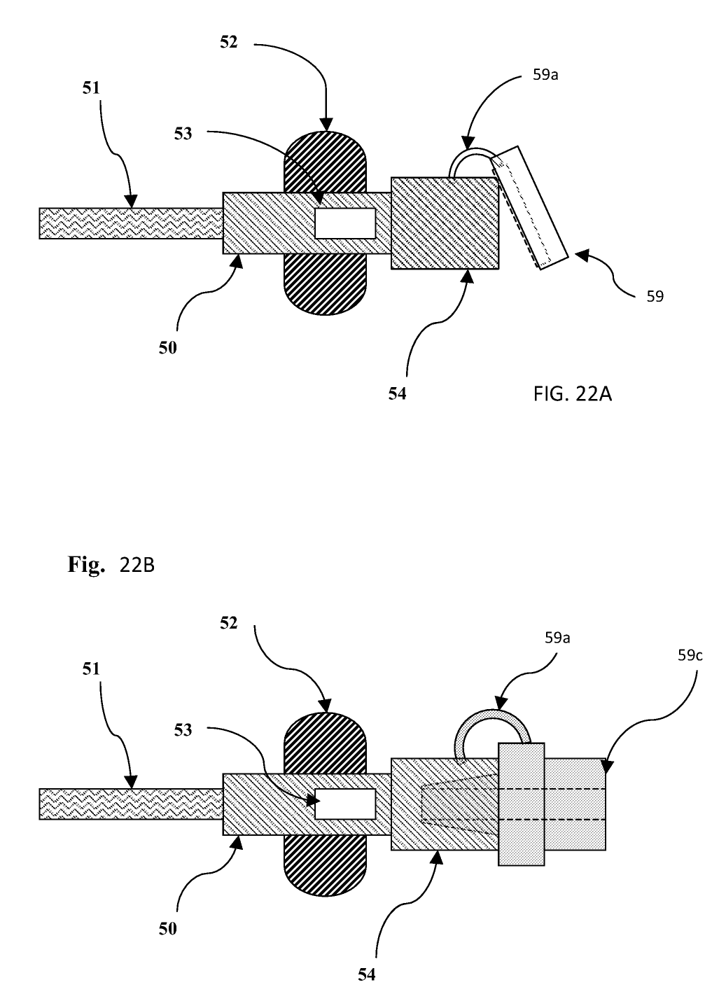

The embodiment in FIG. 22A shows the hub 50, cannula 51, stabilizing tabs 52, flash window 53, hub 54, and a cover 59 (e.g., removable cover 59) that can be snapped or pushed over the open second end of the hub 54 to help keep it sterile. In some embodiments, the cover 59 may be attached to the hub 54 by an attachment means 59a (e.g., a flexible plastic strap, tether, link, or the like) so that it is readily available for the care provider. The embodiment in FIG. 22B shows a saline lock 59c attached to the hub 54 via an attachment means 59a (e.g., a flexible plastic strap, tether, link, or the like), again to enable easy access to the saline lock 59c and also allow a care provider to use one hand to install the saline lock 59c into the device. Both embodiments in FIG. 22A and FIG. 22B may be used with or without a valve (e.g., reed-type valve 26, spring-tensioned clapper-type valve 46, tines valve 36, compressible valve 1150, etc.) in the catheter.

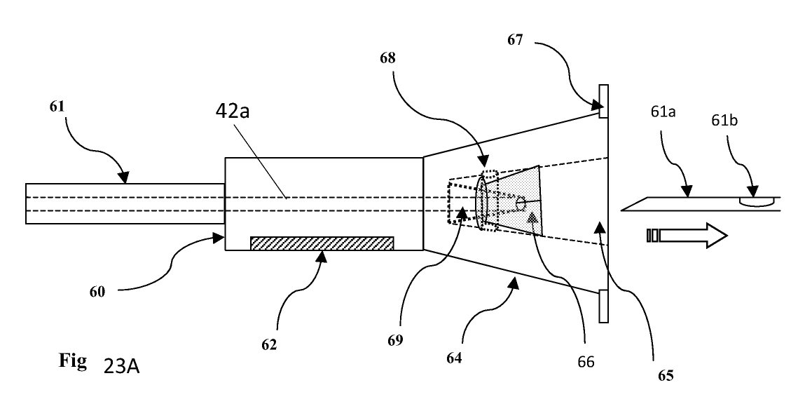

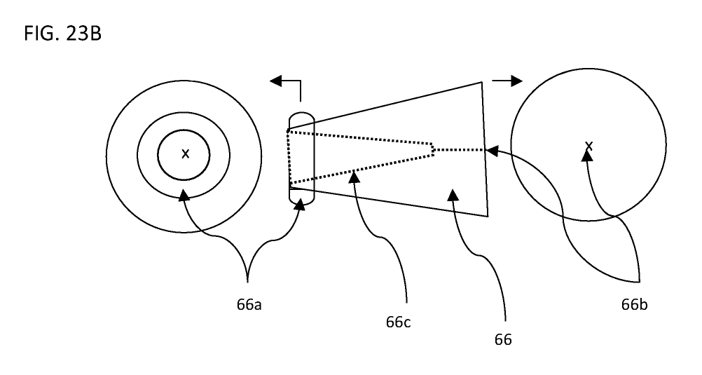

The embodiment in FIG. 23A shows a hub 60 (e.g., a semitransparent hub 60), a cannula 61, a stabilizing tabs 62, a hub 64 with an open second end 65, and a restricted septum-type valve 66 disposed in the hub 64. In some embodiments, a channel 42a is disposed in the hub 60 and is fluidly connected to the cannula 61. In some embodiments, a stationary cone 69 is disposed in the hub 64, and the septum valve 66 is attached to the stationary cone 69 via a mounting component 68 (e.g., a molded ridge). In some embodiments, the septum valve 66 closes when the needle 61a (e.g., optionally with a flash opening 61b) is removed (e.g., FIG. 23A shows the valve 66 in the closed position wherein access cannot be gained to the cannula 61 because the valve 66 is covering the cannula 61 opening). In some embodiments, the septum valve 66 can be opened (moved to the open position) when it is compressed, for example when a male fitting is inserted or locked into the hub (optionally utilizing locking tabs 67 on the second end 65 of the hub 64). The male end is pressed against the septum valve, which in turn forces the septum valve to compress and ride over the stationary cone 69.

FIG. 23B shows a side and detailed view of the septum valve 66 of FIG. 23A. A ridge 66a helps retain the septum valve 66 when in the device. Also shown is a recessed area or cavity 66c disposed in the septum valve 66 adapted to accept the stationary anchor 69 (also stationary anchor 1130) in the hub 64. A slit or small hole 66b is disposed ion the septum valve 66. The slit or hole 66b allows the fluidic connectivity when opened (e.g., when the valve 66 is compressed, fluid access to the cannula can be obtained via the hole 66b.

The septum valve may be constructed from a material comprising a biologically safe elastomeric material or combinations of materials such as, but not restricted to, silicone, rubber or polyurethane so that the material returns to its original shape after it is compressed over the stationary anchor 69 and then released or allowed to relax (e.g., once a syringe, luer lock type device, or other medical connection is removed thus allowing needle-less access or fluid connectivity to the catheter and patient using standard medical devices, such as but not limited to, a needle-less syringe or IV tubing connections).

Details in the various embodiments such as how the devices are molded of plastic and components are joined together via glue or RF welding or assembled with lumen cannula over the needle and how the needle is inserted into the plastic injection molded body of the catheter are all well known in the art.

The term “valve” refers to and includes all types of valves that could be incorporated into the catheter housing to stop the flow of blood back out of the catheters exposed end. Samples of these are spring-loaded clapper valves, spring-loaded ball valves, constant tension activated valves, reed valves, pierceable membranes, self-sealing membranes, compressible septum valves such as those used to seal vials. These are non-limiting examples of various valves or liquid containment mechanisms that would allow repeated access to a patient' s blood without allowing the blood to spill or flow back out of the catheter unless desired. These various examples including those that are generally well known in the art are not meant to limit the scope of the invention in any way.

The term needle stick safety device, refers to any component, mechanism or attachment to the catheter designed to prevent accidental needle sticks once the needle is pulled out of or removed from the catheter, there are many examples of these that are well known in the art.

In the description of the preferred embodiments, reference is made to the accompanying drawings, which form a part hereof, and in which is shown by way of illustration specific embodiments in which the invention may be practiced. In this regard, directional terminology, such as “upper”, “lower”, “side”, “top,” “bottom,” “front,” “back,” “leading,” “trailing,” etc., is used with reference to the orientation of the Figures being described. Because components of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

The reference numbers recited in the below claims are solely for ease of examination of this patent application, and are exemplary, and are not intended in any way to limit the scope of the claims to the particular features having the corresponding reference numbers in the drawings.

1. An angiocatheter system (100) comprising:
(a) a hub (110) having a side wall (112), an inner cavity (113) accessible via an open top end (114), and a bottom portion (115), the inner cavity (113) is adapted to accept a secondary device (101), a centered first hole (119) is disposed in a bottom surface (113a) of the inner cavity (113);
(b) a chamber (120) disposed in the bottom portion (115) of the hub (110) below the bottom surface (113a) of the inner cavity (113), the chamber (120) has a side wall surface (121), a top surface (123), and a bottom surface (124), a second hole (129) is disposed in the bottom surface (124), the second hole (129) is aligned with the first hole (119) of the bottom surface (113a) of the inner cavity (113) of the hub (110);
(c) a catheter (210) fluidly connected to the second hole (129) of the chamber (120) extending through the bottom portion (115) of the hub (110) a distance past the bottom portion (115) of the hub (110);
(d) an anti-leak component (130) housed in the chamber (120), the anti-leak component (130) has a first end (131), a second end (132) opposite the first end (131), a top surface (133), and a bottom surface (134), the top surface (133) is slidably in contact with the top surface (123) of the chamber (120), the bottom surface (134) is slidably in contact with the bottom surface (124) of the chamber (120), a channel (138) is disposed in the anti-leak component (130) extending from the top surface (133) to the bottom surface (134), wherein the anti-leak component (130) is slidable between at least a closed position and an open position, in the closed position the channel (138) is un-aligned with the first hole (119) in the bottom surface (113a) of the inner cavity (113) of the hub (110) and the second hole (129) in the chamber (120), in the open position the channel (138) is aligned with the first hole (119) in the bottom surface (113a) of the inner cavity (113) of the hub (110) and the second hole (129) in the chamber (120), when the anti-leak component (130) is in the open position fluid can flow between the catheter (210) and the inner cavity (113) of the hub (110), the anti-leak component (130) is biased in the closed position via a biasing mechanism; and
(e) a resetting mechanism for moving the anti-leak component (130) to the open position.

2. The system (100) of claim 1, wherein the biasing mechanism functions to push the anti-leak component (130) to un-align the channel (138) with the first hole (119) and second hole (129).

3. The system (100) of claim 1, wherein the biasing mechanism comprises a spring (140) that functions to push the anti-leak component (130) away from the side wall surface (121) of the chamber (120) thereby un-aligning the channel (138) with the first hole (119) and the second hole (129).

4. The system (100) of claim 1, wherein the resetting mechanism comprises a pivot lever (150) disposed in a pivot lever cavity (155) disposed in the side wall (112) of the hub (110), the pivot lever (150) has a first end (151) and a second end (152), the first end (151) is adapted to engage the inner cavity (113) of the hub (110), the second end (152) is adapted to engage the second end (132) of the anti-leak component (130), the pivot lever (150) can pivot between a first position and a second position, in the first position the first end (151) is at least partially pressed into the inner cavity (113) of the hub (110) via an entrance (158) disposed in a inner surface (113b) of the inner cavity (113), the entrance (158) being either a membrane or a hole, in the second position the second end (152) of the pivot lever (150) presses against the second end (132) of the anti-leak component (130) thereby moving the anti-leak component (130) to the open position, the pivot lever (150) is biased in the first position caused by the spring (140) biasing the anti-leak component (130) to the closed position.

5. The system (100) of claim 1, wherein the resetting mechanism is activated upon insertion of a secondary device (101) into the inner cavity (113) of the hub (110).

6. The system (100) of claim 4, wherein insertion of a secondary device (101) into the inner cavity (113) of the hub (110) moves the pivot lever (150) to the second position, which moves the anti-leak component to the open position.

7. The system (100) of claim 1, wherein the resetting mechanism comprises a button (160) disposed on the outer surface of the hub (110).

8. The system (100) of claim 7, wherein the button (160) engages the second end (132) of the anti-leak component (130), when the button (160) is pressed the anti-leak component (130) is moved to the first position.

9. The system (100) of claim 7, wherein the button (160) can be locked in the pressed position via a locking system (250).

10. The system (100) of claim 1, wherein the resetting mechanism comprises a wedge (170) having a pointed end, a flat end, and a hypotenuse end, the pointed end is disposed in the chamber (120) and the hypotenuse end contacts the second end (132) of the anti-leak component (130), the flat end can extend upwardly through the bottom surface (113a) of the inner cavity (113) of the hub (110), the wedge (170) can move between a first position and a second position, in the first position the wedge (170) is pushed upwardly into the inner cavity (113) of the hub (110) via the pressure of the spring (140) and the anti-leak component (130) such that the anti-leak component (130) occupies the closed position, in the second position the wedge (170) is pushed downwardly into the chamber (120) and the hypotenuse end of the wedge (170) presses against the second end (132) of the anti-leak component (130) thereby moving the anti-leak component (130) to the open position, the wedge (170) is biased in the first position caused by the spring (140) biasing the anti-leak component (130) in the closed position.

11. The system (100) of claim 10, wherein the wedge (170) is compressible.

12. The system (100) of claim 10 wherein insertion of a secondary device (101) into the inner cavity (113) of the hub (110) pushes the wedge (170) to the second position, which moves the anti-leak component to the open position.

13. The system (100) of claim 1 further comprising a needle (103) removably housed in the catheter (210), the needle (103) can be withdrawn from the catheter (210).

14. The system (100) of claim 1 further comprising a needle (103) removably housed in the catheter (210), the needle can be withdrawn from the catheter (210), wherein removal of the needle (103) results in the anti-leak valve moving to the closed position to prevent flow from the catheter (210) to the inner cavity (113) of the hub (110).

15. The system (100) of claim 1 further comprising a spacer component (240) disposed on at least a portion of the bottom surface (113a) of the inner cavity (113) of the hub (110).

16. An angiocatheter system (100) comprising:
(a) a hub (110) having an inner cavity (113) accessible via an open top end (114), and a bottom portion (115), the inner cavity (113) is adapted to accept a secondary device (101), a centered first hole (119) is disposed in a bottom surface (113a) of the inner cavity (113);
(b) a catheter (210) extending from a second hole (129) in the bottom portion (115) of the hub (110) a distance past the bottom portion (115) of the hub (110), the first hole (119) and the second hole (129) are aligned;
(c) an anti-leak component (130) housed in the bottom portion (115) of the hub (110), the an anti-leak component (130) can move between at least a closed position and an open position, in the closed position fluid is prevented from flowing between the catheter (210) and the first hole (119) in the hub (110), in the open position fluid can flow between the catheter (210) and the first hole (119) in the hub (110), the anti-leak component (130) is biased in the closed position via a biasing mechanism; and
(e) a resetting mechanism for moving the anti-leak component (130) to the open position.

17. The system (100) of claim 16 further comprising a chamber (120) disposed in the bottom portion (115) of the hub (110) below the bottom surface (113a) of the inner cavity (113), the chamber (120) has a side wall surface (121), a top surface (123), and a bottom surface (124), the second hole (129) is disposed in the bottom surface (124), wherein the anti-leak component (130) is housed in the chamber (120), the anti-leak component (130) comprises a channel (138) extending from a top surface (133) to a bottom surface (134), the top surface (133) of the anti-leak component (130) is slidably in contact with the top surface (123) of the chamber (120), the bottom surface (134) of the anti-leak component (130) is slidably in contact with the bottom surface (124) of the chamber (120), wherein in the closed position the channel (138) is un-aligned with the first hole (119) in the bottom surface (113a) of the inner cavity (113) of the hub (110) and the second hole (129) in the chamber (120), in the open position the channel (138) is aligned with the first hole (119) in the bottom surface (113a) of the inner cavity (113) of the hub (110) and the second hole (129) in the chamber (120).

18. The system (100) of claim 16, wherein the biasing mechanism comprises a spring (140) that pushes the anti-leak component away from the side wall surface (121) of the chamber (120) thereby un-aligning the channel (138) with the first hole (119) and the second hole (129).

19. The system (100) of claim 16, wherein the resetting mechanism comprises a pivot lever (150) disposed in the side wall (111) of the hub (110), the pivot lever (150) has a first end (151) and a second end (152), the first end (151) is adapted to engage the inner cavity (113) of the hub (110), the second end (152) is adapted to engage the second end (132) of the anti-leak component (130), the pivot lever (150) can pivot between a first position and a second position, in the first position the first end (151) is at least partially pressed into the inner cavity (113) of the hub (110) either via a membrane disposed in a inner surface (113b) of the inner cavity (113) or via a side hole disposed in the inner surface (113b) of the inner cavity (113), in the second position the second end (152) of the pivot lever (150) presses against the second end (132) of the anti-leak component (130) thereby moving the anti-leak component (130) to the open position, the pivot lever (150) is biased in the first position caused by the spring (140) biasing the anti-leak component (130) to the closed position.

20. The system (100) of claim 16, wherein the resetting mechanism is activated upon insertion of a secondary device (101) into the inner cavity (113) of the hub (110).

21-45. (canceled)
